(19) United States
(12) Patent Application Publication
(10) Pub. No.: US 2006/0252140 A1
(43) Pub. Date: Nov. 9, 2006

(54) Development of a transposon system for site-specific DNA integration in mammalian cells

(76) Inventors: Stephen R. Yant, Mountain View, CA (US); Mark A. Kay, Los Altos, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/413,481

(22) Filed: Apr. 28, 2006

Related U.S. Application Data
(60) Provisional application No. 60/676,544, filed on Apr. 29, 2005.

(51) Int. Cl. C12N 9/22; C12N 15/74

(52) U.S. Cl. 435/199; 435/455; 435/473

(57) ABSTRACT

The present invention provides a method and compositions for integrating an exogenous nucleic acid into a targeted region of a nucleic acid of a mammalian cell. The compositions include transposase fusion proteins that are adapted to recognize a target site in a nucleic acid. Transposase fusion proteins that include a Sleeping Beauty transposase are provided.

### CROSS-REFERENCE TO RELATED APPLICATIONS

This application claims benefit of U.S. provisional patent application Ser. No. 60/676,544, filed Apr. 29, 2005, which is herein incorporated by reference.

### GOVERNMENT RIGHTS IN THIS INVENTION

The U.S. Government has a paid-up license in this invention and the right in limited circumstances to require the patent owner to license others on reasonable terms as provided for by the terms of grant number DK49022 awarded by the National Institutes of Health (NIH) and of grant number P01 AR44012-07 awarded by the NIH.

### BACKGROUND OF THE INVENTION

1. Field of the Invention

Embodiments of the present invention generally relate to transposases. More particularly, embodiments of the present invention relate to a method of site-specific DNA integration mediated by transposase fusion proteins.

2. Description of the Related Art

Introducing an exogenous nucleic acid into a cell or organism is a frequently used step in basic and applied biological research applications. Many successful methods have been developed to introduce an exogenous nucleic acid into a cell, such as methods that chemically or electrically modify the properties of the cell membrane or cell wall such that the cell is permeable to the exogenous nucleic acid.

However, successful introduction of an exogenous nucleic acid into a cell or organism does not ensure that the exogenous nucleic acid will be expressed in the cell or organism. Nevertheless, methods have been developed to express exogenous nucleic acids in the cell or organism to which they have been transferred. For example, the exogenous nucleic acid may be introduced into the cell or organism on a plasmid that includes a constitutive or inducible promoter that drives expression of the exogenous nucleic acid.

One problem with many currently used methods of expressing an exogenous nucleic acid in a cell or organism is that the expression of the exogenous nucleic acid may continue for a period of time and then stop. For example, the plasmid or vector carrying the nucleic acid may be lost during replication of the host cell. Thus, it is often desirable to introduce an exogenous nucleic acid into a cell such that the exogenous nucleic acid is incorporated into the cell's genome, where it should be maintained throughout many, if not all, subsequent rounds of cell division.

Viral-based vectors, such as retroviral-based vectors, have been developed to introduce an exogenous nucleic acid into a cell such that the exogenous nucleic acid is incorporated into the cell's genome. However, there are significant safety concerns regarding the use of vectors that contain viral sequences, including the triggering of an immune response or the potential generation of a replication-competent virus.

Transposons provide a viable alternative to viral-based vectors for introducing an exogenous nucleic acid into a cell such that the exogenous nucleic acid is incorporated into the cell's genome and for providing stable expression of the exogenous nucleic acid. Transposons are mobile genetic elements found in a variety of species. Transposons typically contain a single gene encoding a transposase protein that binds specifically to short direct repeat sequences (DRs) contained within flanking terminal inverted repeats (IRs). These protein-DNA interactions initiate the excision of the transposon by the transposase from one region of a nucleic acid and results in re-insertion of the transposon into another region of a nucleic acid.

Transposons can be used for biological research and gene therapy applications by replacing the transposase gene between the terminal repeat sequences with an exogenous nucleic acid, such as a gene of interest, and providing a transposase from a separate source, such as another plasmid, to integrate the modified transposon into a genome.

While it has been observed that there are “hotspots” in given nucleic acids in which different transposons tend to integrate, it is difficult to predict the site of insertion of a transposon in a genome. Thus, while transposons may be used to stably express an exogenous nucleic acid in a cell, the apparently random or at least unpredictable insertion of the exogenous nucleic acid into the genome may cause a deleterious up-regulation or down-regulation of a neighboring gene, as has been observed during the integration of retroviral vectors in both mice and humans.

Thus, there is presently a tremendous need for methods that enable targeted, predictable, and/or site-specific integration of an exogenous nucleic acid into a genome, especially without the use of viral-based components.

### SUMMARY OF THE INVENTION

The present invention generally provides methods and compositions for site-specific integration of an exogenous nucleic acid into a genome. In particular, a method of integrating an exogenous nucleic acid into the genome of a mammalian cell using a transposase fusion protein is provided. In one embodiment, a method comprises introducing a Sleeping Beauty transposon comprising an exogenous nucleic acid and a source of Sleeping Beauty transposase activity into the mammalian cell and integrating the exogenous nucleic acid into a targeted region of the genome of the mammalian cell.

In another embodiment, a source of transposase activity that is adapted to recognize a targeted region of the genome and integrate an exogenous nucleic acid from a transposon into the targeted region of the genome is provided. The source of the transposase activity may include a transposase fusion protein. The transposase fusion protein may include a site-specific DNA binding protein that can recognize a specific nucleic acid sequence and direct exogenous nucleic acid integration at or near the site of the specific nucleic acid sequence. In one aspect, the source of transposase activity is a transposase fusion protein comprising a hyperactive Sleeping Beauty transposase mutant fused to the polydactyl zinc finger protein E2C. The polydactyl zinc finger protein E2C of the transposase fusion protein is capable of recognizing a unique site in the genome of a target human cell such that the transposase fusion protein integrates an exogenous nucleic acid from a transposon into or near the unique site.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1A is a schematic diagram of a transposase fusion protein according to an embodiment of the invention.

FIG. 1B is a schematic diagram comparing site-specific and non-site-specific integration events.

FIG. 2A is a schematic diagram of the activator plasmid constructs and the target sites of the report plasmid constructs used in a reporter assay for monitoring DNA-binding specificity of candidate DNA-binding domains (DBD) for fusion proteins provided herein.

FIG. 2B is a series of graphs showing the results of a reporter assay using the constructs of FIG. 2A.

FIG. 3A is a schematic overview of SB transposases.

FIG. 3B is a graph showing the number of G418^{R} colonies obtained from experiments in which HeLa cells were transfected with a neomycin-marked transposon (pT/nori) together with a plasmid encoding the green fluorescent protein (GFP), the prototypical SB10 transposase, a hyperactive transposase mutant (HSB5), or one of 5 different His-tagged HSB5 transposases.

FIG. 4 is a schematic overview of SB transposase fusion proteins according to embodiments of the invention.

FIG. 5A is a graph showing the number of G418^{R} colonies obtained from experiments in which HeLa cells were transfected with a neomycin-marked transposon (pT/nori) together with a plasmid encoding a transposase, no transposase, or a transposase fusion protein according to an embodiment of the invention.

FIG. 5B is a Western blot analysis of HeLa cell extracts comparing the relative expression levels of unfused HSB5 transposase with that of various SB transposase-E2C fusion proteins.

FIG. 5C shows the PCR results of an excision assay testing transposase fusion proteins according to embodiments of the invention.

FIG. 6 is a DNA mobility shift assay showing the DNA binding abilities of a truncated E2C/SB fusion protein (E2C-L5-SB5-N123) and a mutant version, E2C-L5-SB5-G59A-N123, that contains a single amino acid substitution in the DNA-binding domain of the SB portion of the fusion protein.

FIG. 7A is a schematic of a competition assay to monitor the DNA-binding activity of transposase fusion proteins within human cells.

FIG. 7B is a series of graphs showing the results of the competition assay summarized in FIG. 7A.

FIG. 8 is a graph showing the number of G418^{R} colonies obtained from experiments in which HeLa cells were transfected with a neomycin-marked transposon (pT/nori) together with a plasmid encoding GFP, SB10 transposase, HSB5 transposase, or E2C/SB-5 transposase fusion protein, with one plasmid encoding GFP and a limiting amount of one plasmid encoding HSB5 transposase, or with one plasmid encoding E2C/SB-5 transposase fusion protein and a limiting amount of one plasmid encoding HSB5 transposase.

FIG. 9A is a schematic drawing of a donor plasmid and a target plasmid used in a transposition assay according to an embodiment of the invention.

FIG. 9B is a list summarizing the steps of a method of isolating and characterizing individual transposition events according to an embodiment of the invention.

FIG. 9C is a graph showing the distribution of transposon integration mediated by the E2C/SB-5 transposase fusion protein into different sites of target plasmids.

FIG. 10A is a schematic drawing of a donor plasmid for use in a transposition assay according to an embodiment of the invention.

FIG. 10B is a schematic drawing of target plasmids for use in a transposition assay according to an embodiment of the invention.

FIG. 10C is a schematic drawing of helper plasmids for use in a transposition assay according to an embodiment of the invention.

FIG. 11 is a schematic diagram showing the plasmids for and steps of a transposition assay according to an embodiment of the invention.

FIG. 12A shows a target plasmid for a transposition assay according to an embodiment of the invention.

FIG. 12B shows the results of a DNA blot analysis of targeted integration achieved in an assay using the plasmids of FIG. 11 and 12A.

FIG. 12C is a graph showing the targeted transposition frequencies provided by different helper plasmids encoding different transposase proteins in a transposition assay performed using the target plasmid of FIG. 12A.

FIG. 13A is schematic diagram illustrating the differences in the binding of a transposase fusion protein comprising the 6 zinc fingers of E2C to a mutant e2C site and a canonical, i.e., non-mutant, e2C site.

FIG. 13B is a diagram illustrating a transposasome tether and a transposon tether according to embodiments of the invention.

FIG. 14A illustrates a method of using the transposase fusion proteins provided according to embodiments of the invention to mediate site-specific integration in the human genome

FIG. 14B is a schematic overview of a method to map transposon integrations in the human genome performed according to embodiments of the invention.

FIG. 14C is a schematic diagram of human chromosomes that shows the distribution of E2C-L5-SB insertion sites in the human genome from transpositions performed according to embodiments of the invention.

### DETAILED DESCRIPTION

Embodiments of the present invention generally provide a method of site-specific DNA integration mediated by transposases. Embodiments of the present invention also provide transposase fusion proteins, such as Sleeping Beauty (SB) transposase fusion proteins, that direct site-specific DNA integration. As defined herein, a transposase fusion protein is a protein comprising the amino acid sequence of a transposase (or of at least a portion of a transposase having transposase activity) and the amino acid sequence of one or more other proteins (or at least of a portion of one or more other proteins). The transposase fusion protein may also comprise other amino acids, such as amino acids that provide a flexible linker region between the transposase and other protein domains of the fusion protein such that the transposase fusion protein is capable of folding properly and retains activity.

Embodiments of the invention provide a method of integrating an exogenous nucleic acid into another nucleic acid, such as a nucleic acid of a mammalian cell. For example, in one embodiment, an exogenous nucleic acid located between the terminal repeats of a transposon and a source of transposase activity, such as a fusion protein comprising a transposase fused to a heterologous DNA-binding protein, are introduced into a mammalian cell. The exogenous nucleic acid and the source of transposase activity may be introduced into the cell in vitro or in vivo. The transposase fusion protein recognizes a targeted region of the nucleic acid in the cell and facilitates the integration of the exogenous nucleic acid into or near the targeted region. The targeted region may be in the genome of the cell or on a plasmid.

The source of transposase activity may be a fusion protein comprising a transposase and a site-specific DNA binding protein, such as a site-specific zinc-finger DNA binding protein. The site-specific DNA binding protein provides site-specific integration capability to the transposase fusion protein since the site-specific DNA binding portion of the fusion protein can recognize a specific nucleic acid sequence and direct exogenous nucleic acid integration at or near the site of the specific nucleic acid sequence. An exogenous nucleic acid may be targeted to different sites in a genome by selecting site-specific DNA binding proteins that recognize different target sites in a genome and creating different fusion proteins comprising site-specific DNA binding proteins that have different target site specificities.

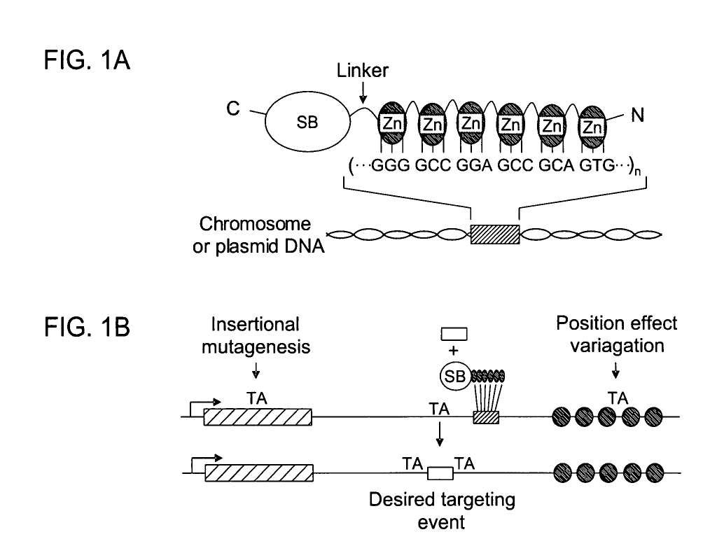

Certain embodiments of the invention provide a fusion protein comprising the hyperactive Sleeping Beauty transposase HSB5 and the polydactyl zinc finger protein E2C and will be described further below. A brief summary of embodiments of fusion proteins comprising a SB transposase and zinc fingers will be provided herein with respect to FIGS. 1A and 1B.

FIG. 1A is a schematic diagram of a fusion protein comprising six zinc fingers (Zn) connected to the SB transposase by a flexible peptide linker. The N and C termini of the protein are indicated. Each Zn finger makes contact with three consecutive base pairs in a recognition sequence. The recognition sequence shown is a DNA substrate containing the canonical binding site for E2C, 5′-GGG GCC GGA GCC GCA GTG (SEQ ID NO: 1), in various numbers (n) within the context of a target plasmid or host cell chromosome or genome.

FIG. 1B illustrates the potential advantages of site-directed or site-specific integration. Transposase proteins of the prior art recognize a TA dinucleotide and thus normally target many sites in the human genome. This can result in undesired targeting events, leading to insertional mutagenesis or attenuated gene expression due to position-effect variegation. Physical linkage of a sequence-specific DNA-binding domain to the transposase protein offers one way to target integration of the transposon (open rectangle) to a single desired site.

While certain embodiments of the invention are described further with respect to a fusion protein comprising the hyperactive Sleeping Beauty transposase HSB5 and the polydactyl zinc finger protein E2C, it is recognized that other transposase fusion proteins comprising other transposases and/or other site-specific DNA binding proteins may be used according to embodiments of the invention. Examples of other transposases (with their associated transposons) that may be used include Himar1, Mos1, Minos, Frog Prince, PiggyBac, Tn5, Tc1 and Tc3. Examples of other site-specific DNA binding proteins that may be used include a human codon-optimized E2C protein, the three zinc finger protein zif268, or one or more of various synthetic 3 to 8 zinc finger proteins that could readily be isolated in the laboratory to bind with high-affinity to pre-specified region(s) of a host cell genome.

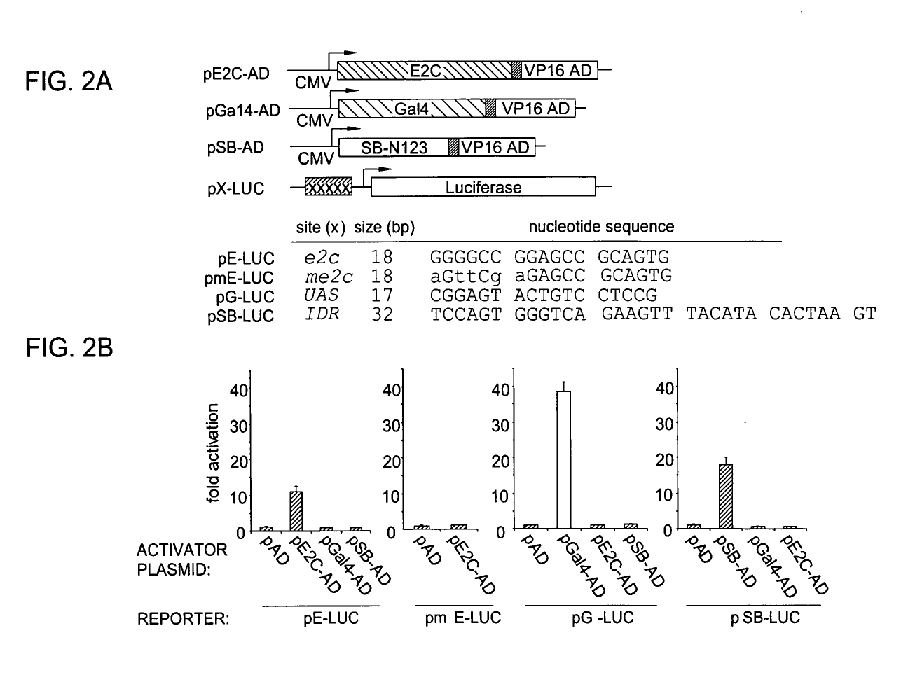

FIGS. 2A and 2B summarize the constructs used and the results obtained in a reporter assay for monitoring DNA-binding specificity of candidate DNA-binding domains (DBD) for the fusion proteins provided herein. Prospective DBDs for use in site-selective DNA-tethering strategies were first fused to the VP16 activation domain and expressed from the strong CMV promoter. The activator plasmids included the following DBDs: E2C, a synthetic polydactyl zinc-finger protein that recognizes a unique site (e2C) on human chromosome 17; Gal4, the DNA-binding domain from the Gal4 protein; and SB-N123, the SB transposase N-terminal 123 amino acid DNA-binding domain. These activator plasmids were co-transfected into HeLa cells together with a reporter plasmid (pX-LUC) containing a luciferase gene, a minimal promoter element, and five upstream binding sites for the DBDs (XXXXX) such that co-delivery of an appropriate activator and reporter plasmid results in activation of the downstream luciferase reporter gene. The upstream binding sites included the following sites: e2C, the E2C binding site; me2C, a mutated e2C control site; UAS, a Gal4 upstream activator sequence; IDR, an inner direct repeat which is a binding site for SB. The sequences of the binding sites are also shown in FIG. 2A. The sequence of the e2C site is GGGGCCGGAGCCGCAGTG (SEQ ID NO: 1). The sequence of the me2C site is AGTTCGAGAGCCGCAGTG (SEQ ID NO: 2). The sequence of the UAS site is CGGAGTACTGTCCTCCG (SEQ ID NO: 3). The sequence of the IDR site is TCCAGTGGGTCAGAAGTTTACATACACTAAGT (SEQ ID NO: 4). FIG. 2B illustrates the DNA-binding specificity of the independent protein domains within the context of human cells. Each graph displays luciferase activity relative to transfection with empty vector (pAD). The bars represent the average (mean±standard deviation) obtained from three independent transfection experiments.

Since codon optimization can be used to increase heterologous gene expression and E2C was isolated from bacteria, we re-synthesized it together with a [(Gly-Gly-Ser)₅] flexible linker using codons optimized for expression in human cells. This human codon-optimized E2C-(Gly-Gly-Ser)₅ gene (hE2C) was fused to HSB5 and was found to be expressed to ˜3-fold higher levels in transfected HeLa cells compared to the non-codon-optimized E2C/SB-5 fusion protein (identical in amino acid sequence). This hE2C/SB-5 fusion protein is expected to support higher integration frequencies.

The nucleotide sequence for the humanized E2C-(Gly-Gly-Ser)₅ gene is as follows:

| | |
|---|---|
| ATGGCACAGGCAGCTCTGGAACCCGGAGAGAAACCTTATGCCTGTC | (SEQ ID NO: 5) |
| CCGAATGTGGTAAGTCCTTTTCTCGAAAAGATAGCCTTGTGAGACACCAGAGAA | |
| CCCATACCGGTGAAAAGCCTTACAAGTGCCCAGAGTGCGGCAAGTCTTTCTCC | |
| CAGTCCGGGGATCTTAGACGGCACCAACGCACCCACACTGGGGAGAAGCCAT | |
| ACAAATGTCCAGAGTGTGGTAAATCCTTCAGCGACTGCCGCGACCTGGCAAGG | |
| CATCAACGCACACATACAGGAGAAAAGCCCTACGCTTGTCCCGAATGCGGTAA | |
| ATCTTTCTCTCAGTCTTCACATCTTGTGAGGCACCAGCGCACACACACCGGGG | |
| AGAAACCATATAAATGTCCTGAATGCGGAAAGTCTTTTAGCGATTGCAGGGATC | |
| TCGCTAGACATCAGCGCACCCACACAGGCGAAAAGCCTTATAAGTGTCCAGAG | |
| TGCGGTAAATCCTTTAGCAGATCCGACAAACTTGTACGACACCAAAGGACCCAT | |
| ACTGGTAAGAAAACAAGCGGTCAGGCAGGAGGAGGTTCTGGCGGCTCCGGAG | |
| GGAGCGGAGGGTCTGGAGGGAGC. | |

Two additional related embodiments are described below. 1) The phiC31 integrase protein has been reported to direct exogenous DNA integration into a smaller subset of potential genomic sites in mammalian cells compared to other integrating vectors, but phiC31-mediated integration is still not “site-specific”. In one embodiment for site-specific integration, a synthetic zinc finger protein is fused to the phiC31 integrase to preferentially direct integrations into only one of the ˜1000 potential computer-predicted target sites. This could be done by pre-selecting a zinc finger protein that can bind specifically to DNA flanking one of these potential target sites. 2) Alternatively, in another embodiment, it may be possible to more efficiently direct integrations into specific target sites by co-expressing unfused HSB5 transposase together with hE2C protein that is fused to only the N-terminal leucine-zipper protein-protein interaction domain of SB10. In this manner, the unfused transposase will retain much greater integration activity but now may preferentially integrate exogenous DNA into predetermined sites via the physical interaction of the two transposase and hE2C-SB-leucine zipper fusion proteins.

SB Transposons and Transposases

The SB transposon is a Tc1/mariner-like transposon that was reconstructed from pieces of defective or inactive transposable elements in fish genomes. The wild-type SB transposon and transposase are described briefly below. The wild-type SB transposon and transposase are further described in commonly assigned U.S. Pat. No. 6,613,752, and U.S. Patent Publication No. 2005/0003542, both of which are herein incorporated by reference.

As defined herein, a Sleeping Beauty transposon is a nucleic acid that is flanked at either end by inverted repeats which are recognized by an enzyme having Sleeping Beauty transposase activity. By “recognized” is meant that a Sleeping Beauty transposase is capable of binding to the inverted repeat and then integrating the transposon flanked by the inverted repeat into the genome of the target cell. Representative inverted repeats that may be found in the Sleeping Beauty transposons include those disclosed in WO 98/40510 and WO 99/25817, both of which are incorporated by reference herein. Of particular interest are inverted repeats that are recognized by the “wild-type” SB10 Sleeping Beauty transposase which has an amino acid identity to SEQ ID NO:6, which is:

(SEQ ID NO: 6)

MGKSKEISQD LRKKIVDLHK SGSSLGAISK
RLKVPRSSVQ
TIVRKYKHHG TTQPSYRSGR RRVLSPRDER TLVRKVQINP
RTTAKDLVKM
LEETGTKVSI STVKRVLYRH NLKGRSARKK PLLQNRHKKA
RLRFATAHGD
KDRTFWRNVL WSDETKIELF GHNDHRYVWR KKGEACKPKN
TIPTVKHGGG
SIMLWCGFAA GGTGALHKID GIMRKENYVD ILKQHLKTSV
RKLKLGRKWV
FQMDNDPKHT SKVVAKWLKD NKVKVLEWPS QSPDLNPIEN
LWAELKKRVR
ARRPTNLTQL HQLCQEEWAK IHPTYCGKLV EGYPKRLTQV
KQFKGNATKY.

A nucleic acid sequence encoding the SB10 Sleeping Beauty Transposase is:

(SEQ ID NO: 7)

ATGGGAAAAT CAAAAGAAAT CAGCCAAGAC
CTCAGAAAAA
AAATTGTAGA CCTCCACAAG TCTGGTTCAT CCTTGGGAGC
AATTTCCAAA
CGCCTGAAAG TACCACGTTC ATCTGTACAA ACAATAGTAC
GCAAGTATAA
ACACCATGGG ACCACGCAGC CGTCATACCG CTCAGGAAGG
AGACGCGTTC
TGTCTCCTAG AGATGAACGT ACTTTGGTGC GAAAAGTGCA
AATCAATCCC
AGAACAACAG CAAAGGACCT TGTGAAGATG CTGGAGGAAA
CAGGTACAAA
AGTATCTATA TCCACAGTAA AACGAGTCCT ATATCGACAT
AACCTGAAAG
GCCGCTCAGC AAGGAAGAAG CCACTGCTCC AAAACCGACA
TAAGAAAGCC
AGACTACGGT TTGCAACTGC ACATGGGGAC AAAGATCGTA
CTTTTTGGAG
AAATGTCCTC TGGTCTGATG AAACAAAAAT AGAACTGTTT
GGCCATAATG
ACCATCGTTA TGTTTGGAGG AAGAAGGGGG AGGCTTGCAA
GCCGAAGAAC
ACCATCCCAA CCGTGAAGCA CGGGGGTGGC AGCATCATGT
TGTGGGGGTG
CTTTGCTGCA GGAGGGACTG GTGCACTTCA CAAAATAGAT
GGCATCATGA
GGAAGGAAAA TTATGTGGAT ATATTGAAGC AACATCTCAA
GACATGAGTC
AGGAAGTTAA AGCTTGGTCG CAAATGGGTC TTCCAAATGG
ACAATGACCC
CAAGCATACT TCCAAAGTTG TGGCAAAATG GCTTAAGGAC
AACAAAGTCA
AGGTATTGGA GTGGCCATCA CAAAGCCCTG ACCTCAATCC
TATAGAAAAT
TTGTGGGCAG AACTGAAAAA GCGTGTGCGA GCAAGGAGGC
CTACAAACCT
GACTCAGTTA CACCAGCTCT GTCAGGAGGA ATGGGCCAAA
ATTCACCCAA
CTTATTGTGG GAAGCTTGTG GAAGGCTACC CGAAACGTTT
GACCCAAGTT
AAACAATTTA AAGGCAATGC TACCAAATAC TAG

Inverted repeats that are recognized by other SB transposases or SB transposase fusion proteins according to embodiments of the invention are also of interest. It is noted that the SB transposase fusion proteins according to embodiments of the invention typically recognize the same inverted repeats recognized by the SB10 transposase.

In many embodiments, each inverted repeat of the transposon includes at least one direct repeat. The transposon element is a linear nucleic acid fragment that can be used as a linear fragment or circularized, for example in a plasmid. In certain embodiments, there are two direct repeats in each inverted repeat sequence. Direct repeat sequences of interest include:

The 5′ outer repeat: 5′-GTTCAAGTCGGAAGTTTACATACACTTAG-3′ (SEQ ID NO:8); the 5′ inner repeat: 5′-CAGTGGGTCAGAAGTTTACATACACTAAGG-3′ (SEQ ID NO:9); the 3′ inner repeat: 5′-CAGTGGGTCAGAAGTTAACATACACTCAATT-3′ (SEQ ID NO:10); the 3′ outer repeat: 5′-AGTTGAATCGGAAGTTTACATACACCTTAG-3′ (SEQ ID NO:11).

A consensus sequence of interest is:

(SEQ ID NO: 12)

5′-CA(GT)TG(AG)GTC(AG)GAAGTTTACATACACTTAAG-3′

In one embodiment, a direct repeat sequence of interest includes at least the following sequence:

ACATACAC (SEQ ID NO:13)

In certain embodiments, the inverted repeat sequence is:

(SEQ ID NO: 14)

5′-AGTTGAAGTC GGAAGTTTAC ATACACTTAA
GTTGGAGTCA
TTAAAACTCG TTTTTCAACT ACACCACAAA TTTCTTGTTA
ACAAACAATA
GTTTTGGCAA GTCAGTTAGG ACATCTACTT TGTGCATGAC
ACAAGTCATT
TTTCCAACAA TTGTTTACAG ACAGATTATT TCACTTATAA
TTCACTGTAT
CACAATTCCA GTGGGTCAGA AGTTTACATA CACTAA-3′.

and a second inverted repeat is:

(SEQ ID NO: 15)

5′-TTGAGTGTAT GTTAACTTCT GACCCACTGG
GAATGTGATG
AAAGAAATAA AAGCTGAAAT GAATCATTCT CTCTACTATT
ATTCTGATAT
TTCACATTCT TAAAATAAAG TGGTGATCCT AACTGACCTT
AAGACAGGGA
ATCTTTACTC GGATTAAATG TCAGGAATTG TGAAAAAGTG
AGTTTAATG
TATTTGGCTA AGGTGTATGT AAACTTCCGA CTTCAACTG-3′.

In certain embodiments, the SB transposon is characterized by the presence of two additional elements as compared to the above described wild-type SB transposon, where the two additional elements provide for enhanced integration efficiency, as measured using the above described assay, either with the SB10 transposase of SEQ ID NO.: 6 or with a transposase fusion protein of the present invention. Specifically, the transposon of these embodiments includes an extra transposon enhancer element (known in the art as an HDR or half direct repeat), e.g., (GTTTACAGACAGA) (SEQ ID NO:16), in addition to the transposon enhancer element found in the wild type left IDR domain. In many embodiments, this additional transposon enhancer element is present in the right flanking IDR domain, e.g., as a duplicate of the wild-type left IDR that has been substituted for the right IDR (as reported in Izsvak et al. J. Biochem. (2002)277(37):34581-8). In addition, the transposon of this embodiment also includes an additional TA dinucleotide adjacent to the right flanking TA dinucleotide (as described in Cui et al., J Mol. Biol. (2002) 318(5):1221-35, which is herein incorporated by reference).

While the SB10 transposase has a high level of transposase activity compared to other known transposases, hyperactive SB transposase mutants have also been developed for use in applications such as gene therapy, where a high level of transposon integration is desired. As defined herein, hyperactive SB transposases are transposases that provide a higher level of integration than the “wild-type” SB10 transposase. Hyperactive SB transposases are described in commonly assigned U.S. Patent Publication No. 2005/0003542.

Embodiments of the invention provide transposase fusion proteins comprising the hyperactive SB transposase mutant HSB5 or a portion thereof, and the polydactyl zinc finger protein E2C. The polydactyl zinc finger protein E2C is a protein that contains 6 zinc finger domains and binds 18 base pairs of contiguous DNA sequence. The polydactyl zinc finger protein E2C and other polydactyl zinc finger proteins are described in U.S. Pat. Nos. 6,140,081 and 6,610,512, both of which are incorporated by reference herein.

The amino acid sequence of the hyperactive SB transposase mutant HSB5 is shown below:

(SEQ ID NO: 17)

MGKSKEISQD LRAKIVDLHK SGSSLGAISK
RLAVPRSSVQ
TIVRKYKHHG TTQPSYRSGR RRVLSPRDER TLVRKVQINP
RTAAKDLVKM
LEETGTKVSI STVKRVLYRH NLKGRSARKK PLLQNRHKKA
RLRFATAHGD
KDRTFWRNVL WSDETKIELF GHNDHRYVWR KKGEACKPKN
TIPTVKHGGG
SIMLWCGFAA GGTGALHKID GIMRKENYVD ILKQHLKTSV
RKLKLGRKWV
FQMDNDPKHT SKVVAKWLKD NKVKVLEWPA QSPDLNPIEN
LWAELKKRVR
ARRPTNLTQL HQLCQEEWAK IHPTYCGKLV EGYPKRLTQV
KQFKGNATKY.

The amino acids in bold type are the four amino acids that differ between the SB10 transposase and the HSB5 transposase.

A nucleic acid sequence encoding the hyperactive SB transposase mutant HSB5 is:

| | |
|---|---|
| ATGGGAAAATCAAAAGAAATCAGCCAAGACCTCAGAGCGAAAATTGT | (SEQ ID NO: 18) |
| AGACCTCCACAAGTCTGGTTCATCCTTGGGAGCAATTTCCAAACGCCTGGCGG | |
| TACCACGTTCATCTGTACAAACAATAGTACGCAAGTATAAACACCATGGGACCA | |
| CGCAGCCGTCATACCGCTCAGGAAGGAGACGCGTTCTGTCTCCTAGAGATGAA | |
| CGTACTTTGGTGCGAAAAGTGCAAATCAATCCCAGAACAGCGGCAAAGGACCT | |
| TGTGAAGATGCTGGAGGAAACAGGCACAAAAGTATCTATATCCACAGTAAAACG | |
| AGTCCTATATCGACATAACCTGAAAGGCCGCTCAGCAAGGAAGAAGCCACTGC | |
| TCCAAAACCGACATAAGAAAGCCAGACTACGGTTTGCAACTGCACATGGGGAC | |
| AAAGATCGTACTTTTTGGAGAAATGTCCTCTGGTCTGATGAAACAAAAATAGAA | |
| CTGTTTGGTCATAATGACCATCGTTATGTTTGGAGGAAGAAGGGGGAGGCTTG | |
| CAAGCCGAAGAACACCATCCCAACCGTGAAGCACGGGGGTGGCAGCATCATG | |
| TTGTGGGGGTGCTTTGCCGCAGGAGGGACTGGTGCACTTCACAAAATAGATGG | |
| CATCATGAGGAAGGAAAATTATGTGGATATATTGAAGCAACATCTCAAGACATC | |
| AGTCAGGAAGTTAAAGCTTGGTCGCAAATGGGTCTTCCAAATGGACAATGACC | |
| CCAAGCATACTTCCAAAGTTGTGGCAAAATGGCTTAAGGACAACAAAGTCAAGG | |
| TATTGGAGTGGCCAGCGCAAAGCCCTGACCTCAATCCTATAGAAAATTTGTGG | |
| GCAGAACTGAAAAAGCGTGTGCGAGCAAGGAGGCCTACAAACCTGACTCAGTT | |
| ACACCAGCTCTGTCAGGAGGAATGGGCCAAAATTCACCCAACTTATTGTGGGA | |
| AGCTTGTGGAAGGCTACCCGAAACGTTTGACCCAAGTTAAACAATTTAAAGGCA | |
| ATGCTACCAAATACTAG. | |

The amino acid sequence of the polydactyl zinc finger protein E2C is shown below:

(SEQ ID NO: 19)

MAQAALEPGE KPYACPECGK SFSRKDSLVR
HQRTHTGEKP
YKCPECGKSF SQSGDLRRHQ RTHTGEKPYK CPECGKSFSD
CRDLARHQRT
HTGEKPYACP ECGKSFSQSS HLVRHQRTHT GEKPYKCPEC
GKSFSDCRDL
ARHQRTHTGE KPYKCPECGK SFSRSDKLVR HQRTHTGKKT
SGQAG.

A nucleic acid sequence encoding the polydactyl zinc finger protein E2C is:

| | |
|---|---|
| ATGGCCCAGGCGGCCCTCGAGCCCGGGGAGAAGCCCTATGCTTGT | (SEQ ID NO: 20) |
| CCGGAATGTGGTAAGTCCTTCAGTAGGAAGGATTCGCTTGTGAGGCACCAGCG | |
| TACCCACACGGGTGAAAAACCGTATAAATGCCCAGAGTGCGGCAAATCTTTTA | |
| GTCAGTCGGGGGATCTTAGGCGTCATCAACGCACTCATACTGGCGAGAAGCCA | |
| TACAAATGTCCAGAATGTGGCAAGTCTTTCAGTGATTGTCGTGATCTTGCGAGGC | |
| ACCAACGTACTCACACCGGGGAGAAGCCCTATGCTTGTCCGGAATGTGGTAAGTCCTT | |
| CTCTCAGAGCTCTCACCTGGTGCGCCACCAGCGTACCCACACGGGTGAAAAACCGTAT | |
| AAATGCCCAGAGTGCGGCAAATCTTTTAGTGACTGCCGCGACCTTGCTCGCCATCAAC | |
| GCACTCATACTGGCGAGAAGCCATACAAATGTCCAGAATGTGGCAAGTCTTTCAGCCG | |
| CTCTGACAAGCTGGTGCGTCACCAACGTACTCACACCGGTAAAAAAACTAGTGGCCAG | |
| GCCGGCTAG. | |

Returning to the SB transposons provided herein, the Sleeping Beauty transposase recognized inverted repeats, as described above, flank an insertion nucleic acid, i.e., a nucleic acid that is to be inserted into a target cell genome, as described in greater detail below. The subject transposons may include a wide variety of insertion nucleic acids, where the nucleic acids may include a sequence of bases that is endogenous and/or exogenous to the mammal or multicellular organism, where an exogenous sequence is one that is not present in the target cell while an endogenous sequence is one that pre-exists in the target cell prior to insertion. Either way, the nucleic acid of the transposon is exogenous to the target cell, since it originates at a source other than the target cell and is introduced into the cell by the subject methods, as described infra. In research applications, the exogenous nucleic acid may be a novel gene whose protein product is not well characterized. In such applications, the transposon is employed to stably introduce the gene into the target cell and observe changes in the cell phenotype in order to characterize the gene. Alternatively, in protein synthesis applications, the exogenous nucleic acid encodes a protein of interest which is to be produced by the cell. In yet other embodiments, e.g., in gene therapy, the exogenous nucleic acid is a gene having therapeutic activity. Another way to refer to the insertion nucleic acid of the transposon is as the “inter-inverted repeat domain” of the transposon. The inter inverted repeat domain of the Sleeping Beauty transposon, i.e., that domain or region of the transposon located or positioned between the flanking inverted repeats, may vary greatly in size. The only limitation on the size of the inverted repeat is that the size should not be so great as to inactivate the ability of the transposon system to integrate the transposon into the target genome. The upper and lower limits of the size of this inter inverted repeat domain may readily be determined empirically by those of skill in the art.

A variety of different features may be present in the inter inverted repeat domain of the Sleeping Beauty transposon of the subject systems. In many embodiments, the inter inverted repeat domain is characterized by the presence of at least one transcriptionally active gene. By transcriptionally active gene is meant a coding sequence that is capable of being expressed under intracellular conditions, e.g. a coding sequence in combination with any requisite expression regulatory elements that are required for expression in the intracellular environment of the target cell whose genome is modified by integration of the transposon. As such, the transcriptionally active genes of the subject vectors typically include a stretch of nucleotides or domain, i.e., an expression module, that includes a coding sequence of nucleotides in operational combination, i.e. operably linked, with requisite transcriptional mediation or regulatory element(s). Requisite transcriptional mediation elements that may be present in the expression module include promoters, enhancers, termination and polyadenylation signal elements, splicing signal elements, etc.

Preferably, the expression module includes transcription regulatory elements that provide for expression of the gene in a broad host range. A variety of such combinations are known, where specific transcription regulatory elements include: SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70promoters, (Levy-Holtzman ,R. and I. Schechter (Biochim. Biophys. Acta (1995) 1263: 96-98) Presnail, J. K. and M. A. Hoy, (Exp. Appl. Acarol. (1994) 18: 301-308)) and the like.

In certain embodiments, the at least one transcriptionally active gene or expression module present in the inter inverted repeat domain acts as a selectable marker. Known selectable marker genes include: the thimydine kinase gene, the dihydrofolate reductase gene, the xanthine-guanine phosporibosyl transferase gene, CAD, the adenosine deaminase gene, the asparagine synthetase gene, antibiotic resistance genes, e.g. tet^{r}, amp^{r}, Cm^{r} or cat, kan^{r} or neo^{r} (aminoglycoside phosphotransferase genes), the hygromycin B phosphotransferase gene, genes whose expression provides for the presence of a detectable product, either directly or indirectly, e.g. β-galactosidase, GFP, and the like.

In many embodiments, the at least one transcriptionally active gene or module encodes a protein that has therapeutic activity for the multicellular organism, where such proteins include, but are not limited to: factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides etc, and the like. The above list of proteins refers to mammalian proteins, and in many embodiments human proteins, where the nucleotide and amino acid sequences of the above proteins are generally known to those of skill in the art.

In addition to the at least one transcriptionally active gene, the inverted repeat domain of the subject transposons also typically include at least one restriction endonuclease recognized site, e.g. restriction site, located between the flanking inverted repeats, which serves as a site for insertion of an exogenous nucleic acid. A variety of restriction sites are known in the art and may be included in the inter inverted repeat domain, where such sites include those recognized by the following restriction enzymes: HindIII, PstI, SalI, AccI, HincII, XbaI, BamHI, SmaI, XmaI, KpnI, SacI, EcoRI, and the like. In many embodiments, the vector includes a polylinker, i.e. a closely arranged series or array of sites recognized by a plurality of different restriction enzymes, such as those listed above.

The subject Sleeping Beauty transposon is generally present on a vector which is introduced into the cell, as described in greater detail below. The transposon may be present on a variety of different vectors, where representative vectors include plasmids, viral based vectors, linear DNA molecules and the like, where representative vectors are described infra in greater detail.

In certain embodiments where the source of transposase is a nucleic acid, the Sleeping Beauty transposon and the nucleic acid encoding the transposase are present on separate vectors, e.g. separate plasmids. In certain other embodiments, the transposase encoding domain may be present on the same vector as the transposon, e.g. on the same plasmid. When present on the same vector, the mutant Sleeping Beauty transposase encoding region or domain is located outside the inter inverted repeat flanked domain. In other words, the transposase encoding region is located external to the region flanked by the inverted repeats, i.e. outside the inter inverted repeat domain described supra. For example, the transposase encoding region is positioned to the left of the left terminal inverted repeat or the right of the right terminal inverted repeat.

The various elements of the Sleeping Beauty transposon system employed in the subject methods, e.g. the vector(s) of the subject invention, may be produced by standard methods of restriction enzyme cleavage, ligation and molecular cloning. Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual(1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

One protocol for constructing the subject vectors includes the following steps. First, purified nucleic acid fragments containing desired component nucleotide sequences as well as extraneous sequences are cleaved with restriction endonucleases from initial sources, e.g. a vector comprising the Sleeping Beauty transposase gene. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g. sequences corresponding to the various elements of the subject vectors, as described above is produced. Where desired, the circular molecules are then amplified in a prokaryotic host, e.g. E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc.) The preparation of a representative Sleeping Beauty transposon system is also disclosed in WO 98/40510 and WO 99/25817.

The subject methods find use in a variety of applications in which it is desired to introduce an exogenous nucleic acid into a target cell, and are particularly of interest where it is desired to express a protein encoded by an expression cassette in a target cell. The subject enhanced Sleeping Beauty Transposon systems may be introduced using either in vitro or in vivo protocols.

As indicated above, the subject systems can be used with a variety of target cells, where target cells are often eukaryotic target cells, including, but not limited to, plant and animal target cells, e.g., insect cells, vertebrate cells, particularly avian cells, e.g., chicken cells, fish, amphibian and reptile cells, mammalian cells, including murine, porcine, ovine, equine, rat, ungulates, dog, cat, monkey, and human cells, and the like.

In the methods of the subject invention, the system components are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Alternatively, where the target cell or cells are part of a multicellular organism, the subject system may be administered to the organism or host in a manner such that the targeting construct is able to enter the target cell(s), e.g., via an in vivo or ex vivo protocol. By “in vivo,” it is meant in the target construct is administered to a living body of an animal. By “ex vivo ”it is meant that cells or organs are modified outside of the body. Such cells or organs are typically returned to a living body. Methods for the administration of nucleic acid constructs are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995), using viral vectors (Monahan, et al, Gene Therapy 4:40-49, 1997; Onodera, et al, Blood 91:30-36, 1998), by uptake of “naked DNA”, and the like. Techniques well known in the art for the transformation of cells (see discussion above) can be used for the ex vivo administration of nucleic acid constructs. The exact formulation, route of administration and dosage can be chosen empirically. (See e.g. Fingl et al., 1975, in “The Pharmacological Basis of Therapeutics”, Ch. 1).

As such, in certain embodiments the vector or vectors comprising the various elements of the enhanced Sleeping Beauty transposon system, e.g. plasmids, are administered to a multicellular organism that includes the target cell, i.e. the cell into which integration of the nucleic acid of the transposon is desired. By multicellular organism is meant an organism that is not a single celled organism. Multicellular organisms of interest include plants and animals, where animals are of particular interest. Animals of interest include vertebrates, where the vertebrate is a mammal in many embodiments. Mammals of interest include; rodents, e.g. mice, rats; livestock, e.g. pigs, horses, cows, etc., pets, e.g. dogs, cats; and primates, e.g. humans. As the subject methods involve administration of the transposon system directly to the multicellular organism, they are in vivo methods of integrating the exogenous nucleic acid into the target cell.

The route of administration of the Sleeping Beauty transposon system to the multicellular organism depends on several parameters, including: the nature of the vectors that carry the system components, the nature of the delivery vehicle, the nature of the multicellular organism, and the like, where a common feature of the mode of administration is that it provides for in vivo delivery of the transposon system components to the target cell(s). In certain embodiments, linear or circularized DNA, e.g. a plasmid, is employed as the vector for delivery of the transposon system to the target cell. In such embodiments, the plasmid may be administered in an aqueous delivery vehicle, e.g. a saline solution. Alternatively, an agent that modulates the distribution of the vector in the multicellular organism may be employed. For example, where the vectors comprising the subject system components are plasmid vectors, lipid based, e.g. liposome, vehicles may be employed, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Alternatively, polylysine based peptides may be employed as carriers, which may or may not be modified with targeting moieties, and the like. (Brooks, A. I., et al. 1998, J. Neurosci. Methods V. 80 p: 137-47; Muramatsu, T., Nakamura, A., and H. M. Park 1998, Int. J. Mol. Med. V. 1 p: 55-62). In yet other embodiments, the system components may be incorporated onto viral vectors, such as adenovirus derived vectors, sindbis virus derived vectors, retroviral derived vectors, hybrid vectors, and the like. The above vectors and delivery vehicles are merely representative. Any vector/delivery vehicle combination may be employed, so long as it provides for in vivo administration of the transposon system to the multicellular organism and target cell.

Because of the multitude of different types of vectors and delivery vehicles that may be employed, administration may be by a number of different routes, where representative routes of administration include: oral, topical, intraarterial, intravenous, intraperitoneal, intramuscular, etc. The particular mode of administration depends, at least in part, on the nature of the delivery vehicle employed for the vectors which harbor the Sleeping Beauty transposons system. In many embodiments, the vector or vectors harboring the Sleeping Beauty transposase system are administered intravascularly, e.g. intraarterially or intravenously, employing an aqueous based delivery vehicle, e.g. a saline solution.

In practicing the subject methods, the elements of the Sleeping Beauty transposase system, e.g. the Sleeping Beauty transposon and the Sleeping Beauty transposase source, are introduced into a target cell of the multicellular organism under conditions sufficient for excision of the inverted repeat flanked nucleic acid from the vector carrying the transposon and subsequent integration of the excised nucleic acid into the genome of the target cell. As the transposon is introduced into the cell under conditions sufficient for excision and integration to occur, the subject method further includes a step of ensuring that the requisite Sleeping Beauty transposase activity is present in the target cell along with the introduced transposon. Depending on the structure of the transposon vector itself, i.e. whether or not the vector includes a region encoding a product having Sleeping Beauty transposase activity, the method may further include introducing a second vector into the target cell which encodes the requisite transposase activity, where this step also includes an in vivo administration step.

The amount of vector nucleic acid comprising the transposon element, and in many embodiments, the amount of vector nucleic acid encoding the transposase, that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g. the particular in vivo administration protocol that is employed.

For in vivo administration applications, the particular dosage of each component of the system that is administered to the multicellular organism varies depending on the nature of the transposon nucleic acid, e.g. the nature of the expression module and gene, the nature of the vector on which the component elements are present, the nature of the delivery vehicle and the like. Dosages can readily be determined empirically by those of skill in the art. For example, in mice where the Sleeping Beauty Transposase system components are present on separate plasmids which are intravenously administered to a mammal in a saline solution vehicle, the amount of transposon plasmid that is administered in many embodiments typically ranges from about 0.5 to 40 and is typically about 25 μg, while the amount of Sleeping Beauty transposase encoding plasmid that is administered typically ranges from about 0.5 to 25 and is usually about 1 μg.

Once the vector DNA has entered the target cell in combination with the requisite transposase, the nucleic acid region of the vector that is flanked by inverted repeats, i.e. the vector nucleic acid positioned between the Sleeping Beauty transposase recognized inverted repeats, is excised from the vector via the provided transposase and inserted into the genome of the targeted cell. Introduction of the vector DNA into the target cell is followed by subsequent transposase mediated excision and insertion of the exogenous nucleic acid carried by the vector into the genome of the targeted cell.

The subject methods may be used to integrate nucleic acids of various sizes into the target cell genome. Generally, the size of DNA that is inserted into a target cell genome using the subject methods ranges from about 0.5 kb to 10.0 kb, usually from about 1.0 kb to about 8.0 kb.

The subject methods result in stable integration of the nucleic acid into the target cell genome. By stable integration is meant that the nucleic acid remains present in the target cell genome for more than a transient period of time, and is passed on a part of the chromosomal genetic material to the progeny of the target cell.

The subject methods of stable integration of nucleic acids into the genome of a target cell find use in a variety of applications in which the stable integration of a nucleic acid into a target cell genome is desired. Applications in which the subject vectors and methods find use include: research applications, polypeptide synthesis applications and therapeutic applications.

The subject transposon systems may be used to deliver a wide variety of therapeutic nucleic acids. Therapeutic nucleic acids of interest include genes that replace defective genes in the target host cell, such as those responsible for genetic defect based disease conditions; genes which have therapeutic utility in the treatment of cancer; and the like. Specific therapeutic genes for use in the treatment of genetic defect based disease conditions include genes encoding the following products: factor VII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides etc, and the like. The above list of proteins refers to mammalian proteins, and in many embodiments human proteins, where the nucleotide and amino acid sequences of the above proteins are generally known to those of skill in the art. Cancer therapeutic genes that may be delivered via the subject methods include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like. The subject methods can be used to not only introduce a therapeutic gene of interest, but also any expression regulatory elements, such as promoters, and the like, which may be desired so as to obtain the desired temporal and spatial expression of the therapeutic gene.

In certain embodiments the subject methods may be used for in vivo gene therapy applications. By in vivo gene therapy applications is meant that the target cell or cells in which expression of the therapeutic gene is desired are not removed from the host prior to contact with the transposon system. In contrast, vectors that include the transposon system are administered directly to the multicellular organism and are taken up by the target cells, following which integration of the gene into the target cell genome occurs.

Also provided by the subject invention are kits for use in practicing the subject methods of nucleic acid delivery to target cells. The subject kits generally include one or more components of the subject Sleeping Beauty Transposase systems, which components may be present in an aqueous medium. The subject kits may further include an aqueous delivery vehicle, e.g. a buffered saline solution, etc. In addition, the kits may include one or more restriction endonucleases for use in transferring a nucleic acid into the vector components of the kits. In the subject kits, the above components may be combined into a single aqueous composition for delivery into the host or separate as different or disparate compositions, e.g., in separate containers. Optionally, the kit may further include a vascular delivery means for delivering the aqueous composition to the host, e.g. a syringe etc., where the delivery means may or may not be pre-loaded with the aqueous composition. In addition to the above components, the subject kits will further include instructions for practicing the subject methods.

In one embodiment, a kit comprises a transposon comprising an exogenous nucleic acid and a source of transposase activity that is adapted to recognized a targeted region of a mammalian genome and integrate the exogenous nucleic acid into the targeted region. The transposon may be a SB transposon, and the source of transposase activity may comprise a fusion protein comprising a SB transposase and a site-specific DNA binding protein. For example, the SB transposase may have the sequence of SEQ ID NO: 17 and the site-specific DNA binding protein may comprise the polydactyl zinc finger protein E2C.

A cell comprising a nucleic acid encoding a transposase fusion protein is provided in another embodiment. The transposase fusion protein may be a SB transposase fusion protein.

It is noted that while specific sequences of preferred SB transposon systems are provided herein, the transposases and other components of the system may have other sequences. In particular, derivatives, e.g., homologues, of the amino acid and nucleotide sequences provided herein are encompassed. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides. “Derivatives” of a protein or an amino acid sequence refers to any isolated protein or chain of amino acid molecules that contains significant sequence similarity to the protein or amino acid sequence or a part thereof.

Further embodiments of the invention are described below in the Experiments section.

### EXPERIMENTS

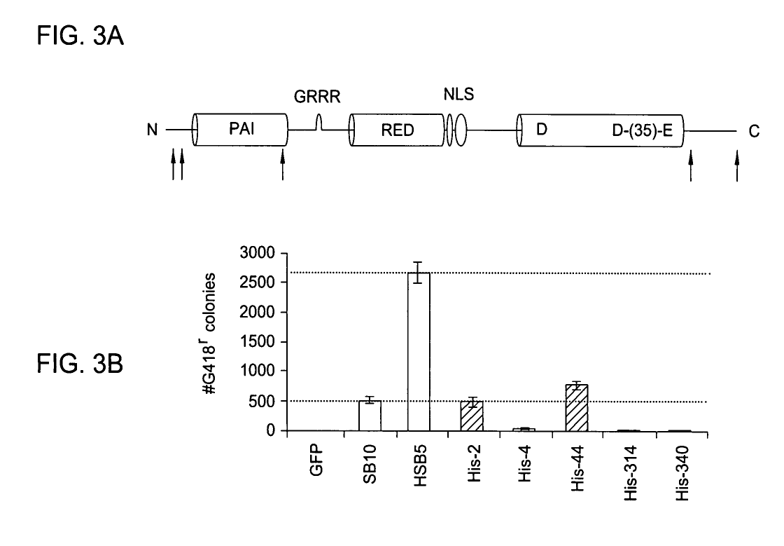

FIG. 3A shows a schematic overview of the SB10 transposase. The SB10 transposase comprises an-N-terminal region having two DNA binding domains and a nuclear localization signal (NLS), and a C-terminal region containing a conserved, D, D-(35)-E catalytic domain.

FIG. 3A also provides a schematic overview of the hyperactive SB transposase mutant HSB5. HSB5 is identical to the SB10 transposase except for 4 amino acid substitutions: K13A/K33A/T83A/S270A. The amino acid sequence of HSB5 is provided above as SEQ ID NO: 17.

Histidine epitope tags (6xHis) were inserted into the HSB5 open reading frame at one of five different sites, as shown schematically by the arrows in FIG. 1A. Two of the sites were terminal (His-2 at the N-terminus and His-340 at the C-terminus) and three of the sites were internal (His-4, His-44, His-314).

The activity of the 6xHis-tagged HSB5 transposases was compared to the activity of the untagged SB10 and HSB5 transposases. HeLa cells were transfected with a neomycin-marked transposon (pT/nori) together with a plasmid encoding GFP, SB10 transposase, HSB5 transposase, or one of the 5 different 6xHis-tagged HSB5 transposases described above. Cells were selected for resistance to the antibiotic G418 for two weeks, at which time individual G418-resistant (G418^{R}) colonies were fixed, stained, and counted. FIG. 3B shows that the SB10 and HSB5 transposases each had significant transposase activity, as estimated by the number of G418 ^{R} colonies. However, three of the 6xHis-tagged HSB5 transposases (His-4, His-314, His-340) had essentially no activity and two of the His tagged HSB5 transposases (His-2, His-44) had approximately 10 fold less activity compared to the untagged HSB5 transposase. Thus, it was found that adding as few as 6 amino acids to a SB transposase can significantly diminish or eliminate its integration activity.

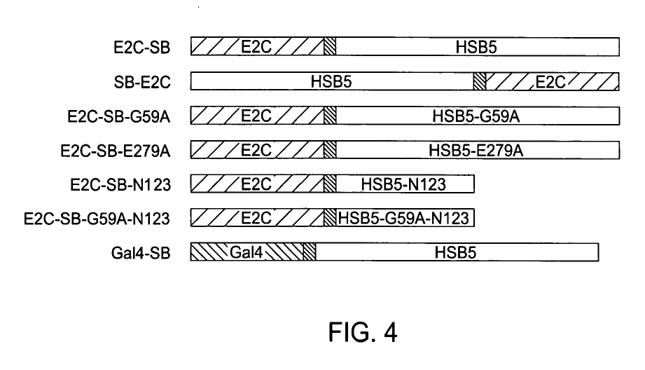

FIG. 4 is a schematic overview of SB transposase fusion proteins according to embodiments of the invention. In one embodiment, the SB transposase fusion protein (E2C-SB) comprises the polydactyl zinc finger protein E2C fused to the N-terminus of the HSB5 transposase with a flexible linker between the polydactyl zinc finger protein E2C and the HSB5 transposase. In another embodiment, the SB transposase fusion protein (SB-E2C) comprises the polydactyl zinc finger protein E2C fused to the C-terminus of the HSB5 transposase with a flexible linker between the polydactyl zinc finger protein E2C and the HSB5 transposase. In either embodiment, the flexible linker may be or include the motif (Gly-Gly-Ser)ₙ, with n=0 to 7. The flexible linker is shown as a black box in FIG. 2A and may have a length of 0-21 amino acids.

Seven subclones containing plasmids encoding an SB transposase fusion protein comprising the polydactyl zinc finger protein E2C fused to the N-terminus of the HSB5 transposase with a flexible linker [(Gly-Gly-Ser)₀₋₇] between the polydactyl zinc finger protein E2C and the HSB5 transposase were selected for analysis and were termed E2C-SB, E2C-(GGS)₁-SB, E2C-(GGS)₃-SB, E2C-(GGS)₄-SB, E2C-(GGS)₅-SB, E2C-(GGS)₆-SB6, and E2C-(GGS)₇-SB. Four subclones containing plasmids encoding an SB transposase fusion protein comprising the polydactyl zinc finger protein E2C fused to the C-terminus of the HSB5 transposase with a flexible linker [(Gly-Gly-Ser)₀₋₃] between the polydactyl zinc finger protein E2C and the HSB5 transposase were selected for analysis and were termed SB-E2C, SB-(GGS)₁-E2C, SB-(GGS)₂-E2C, and SB-(GGS)₃-E2C.

Subclones containing plasmids encoding an SB transposase fusion protein comprising the polydactyl zinc finger protein E2C fused via a flexible linker (Gly-GLy-Ser)₅, i.e., L5, to the N-terminus of two different single amino-acid mutant SB transposases were selected for analysis. One of the transposase fusion proteins comprised a single amino-acid substitution (G59A) in the DNA-binding domain of the HSB5 transposase which disrupts its ability to bind transposon DNA and was termed E2C-L5-SB-G59A, whereas another transposase fusion protein comprised a single amino-acid substitution (E279A) in the catalytic domain of the HSB5 transposase that disrupts its excision and integration activity and was termed E2C-L5-SB-E279A. A transposase fusion protein comprising E2C fused via a flexible linker to the N-terminal 123 amino acids of the HSB5 transposase was termed E2C-SB-N123, and an identical transposase fusion protein except for the single amino-acid substitution (G59A) in the DNA-binding domain of the HSB5 transposase was termed E2C-SB-G59A-N123.

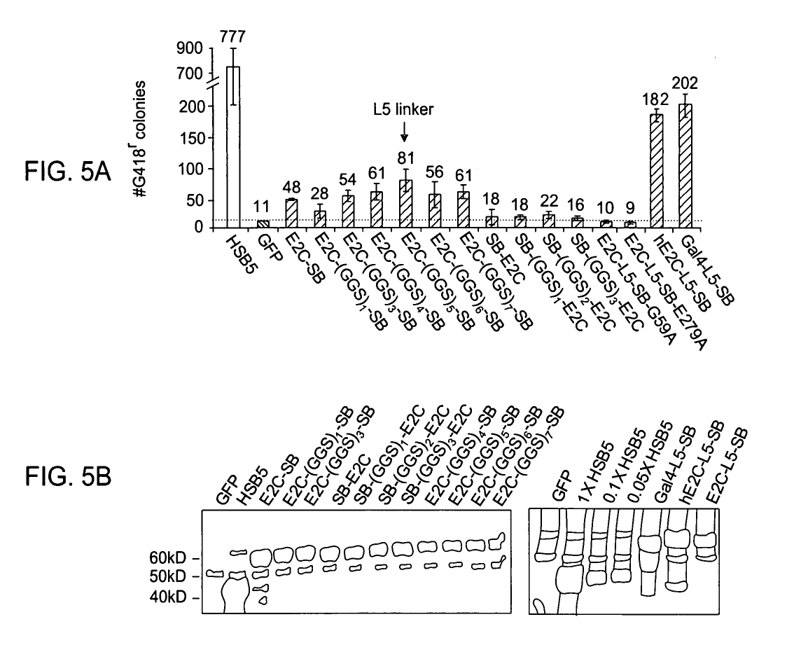

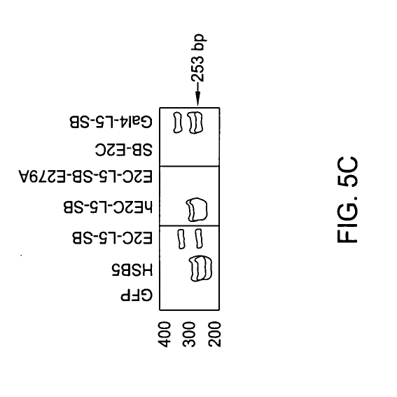

The activity of the transposase fusion proteins was compared to the activity of unfused HSB5 transposase. HeLa cells were transfected with a neomycin-marked (neo^{r}) transposon plasmid together with a plasmid encoding the unfused HSB5 transposase, no transposase (GFP was used as a negative control), or one of the different fusion proteins described above. Transfected cells were growth-selected for two weeks in the antibiotic G418 at 600 μg/ml. Then, all remaining G418^{R} colonies were fixed, stained, and counted to determine relative integration frequencies. The average number of integration events obtained from three independent transfections is shown (mean±standard deviation) in FIG. 5A. The (Gly-Gly-Ser)₅ linker supporting the highest level of integration activity was designated L5. hE2C-L5-SB is the codon-optimized form of E2C for enhanced fusion protein expression in human cells that was described above. FIG. 5A shows that the HSB5 transposase fusion proteins resulted in more G418^{R} colonies than a background level of colonies obtained in the absence of a SB transposase, and thus have transposase activity. FIG. 5A also shows that transposon integration, as estimated by the number of G418^{R} colonies, was completely abrogated by the single amino-acid substitutions in the E2C-L5-SB-G59A and E2C-L5-SB-E279A transposase fusion proteins, which suggests that the integration caused by the other E2C/transposase fusion proteins is SB-mediated.

One probable explanation for the observed lower level of transposase activity of the transposase fusion proteins relative to the unfused transposase HSB5 is that the transposase fusion proteins are not as highly expressed as the unfused transposases. FIG. 5B is a Western blot that shows that significantly less protein was detected by a polyclonal antibody to the SB transposase in cell extracts from cells expressing the fusion proteins relative to cells expressing the HSB5 transposase. Transfected HeLa cells were harvested two days post-transfection, lysed and subjected to immunoblot analysis using a polyclonal antibody against the SB transposase. The right panel shows an attempt to normalize HSB5 and fusion protein expression in the cell by transfecting diminishing amounts of the HSB5 plasmid (1X, 0.1X or 0.05X) relative to the fusion protein constructs.

The excision activity of the fusion proteins was also analyzed. HeLa cells were transfected with a neomycin-marked (neo^{r}) transposon plasmid together with plasmids encoding GFP, HSB5 transposase, or selected fusion proteins. Hirt DNA samples were prepared two days later and used as templates in a series of nested PCR reactions. Transposon excision and subsequent DNA repair by the host enabled the amplification of a diagnostic 253 bp PCR excision-and-repair product. FIG. 5C shows the PCR results.

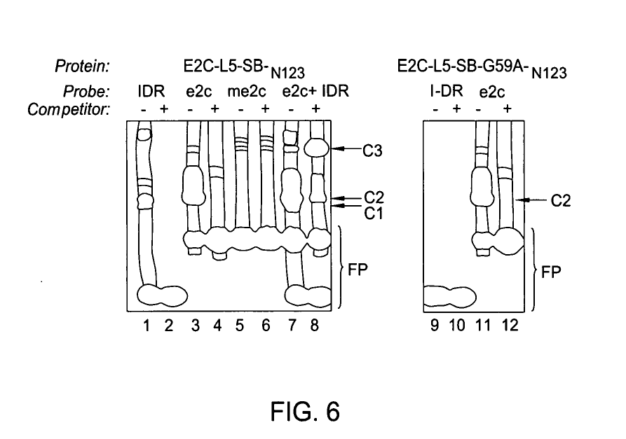

FIG. 6 is a DNA mobility shift assay showing the DNA binding characteristics of a truncated E2C/SB fusion protein (E2C-L5-SB5-N123) and a mutant version of E2C/SB5-N123 (E2C-L5-SB5-G59A-N123) that contains a single amino acid substitution in the DNA-binding domain of the SB portion of the fusion protein. The truncated E2C/SB fusion proteins were produced by in vitro transcription and translation, and then incubated with ³²P-radiolabelled double-stranded DNA probes corresponding to one or a mixture of the following sequences: the SB transposon inner direct repeat (IDR) sequence, the E2C binding site (e2C), and a mutant E2C binding site (mE2C). The E2C binding site is an 18 base pair sequence that is unique in the human genome. 1000-fold excess of competitor was added to some of the complexes as a test for specific DNA binding. Protein/DNA complexes were resolved by electrophoresis through a gel and visualized by autoradiography. Bands corresponding to free probe (FP), fusion protein/IDR complex (C1), fusion protein/e2C complex (C2), and fusion protein/IDR/e2C trimeric complex (C3) are shown in FIG. 6.

FIG. 6 shows that the truncated E2C/SB fusion protein (E2C-L5-SB5-N123) is capable of binding the E2C binding site, the SB transposon inner direct repeat (IDR) sequence, and both the E2C binding site and the SB transposon inner direct repeat (IDR) sequence simultaneously. The truncated E2C/SB fusion protein (E2C-L5-SB5-N123) did not bind to a mutant E2C binding site (mE2C). FIG. 6 also shows that the mutant version of E2C/SB5-N123 (E2C-L5-SB5-G59A-N123) was able to bind the E2C binding site but not the SB transposon inner direct repeat (IDR) sequence.

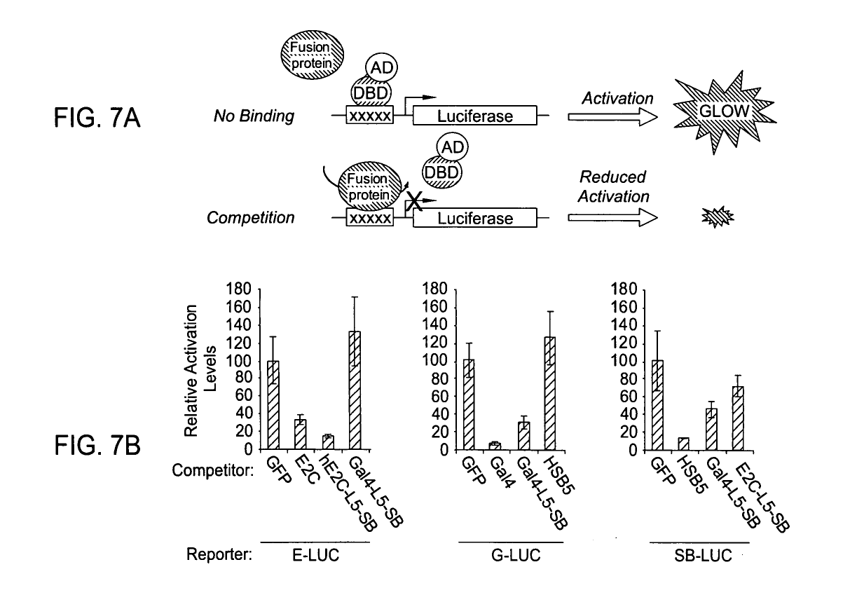

FIG. 7A is a schematic of a competition assay to monitor the DNA-binding activity of full-length E2C-L5-SB and Gal4-L5-SB fusion proteins within human cells. HeLa cells were transfected with luciferase reporter plasmids together with limiting amounts of an activator plasmid encoding their respective trans-activator protein (E2C-AD, Gal4-AD or SB-AD). The reporter plasmid E-LUC includes the e2C site. The reporter plasmid G-LUC includes the Gal4 binding site. The reporter plasmid SB-LUC includes the SB binding site. Cells also received an excess of plasmids encoding various experimental and control proteins to test whether any of the proteins could compete for protein binding at the target sites, thereby reducing the level of luciferase trans-activation in the cell. FIG. 7B shows the results of the competition assay. Each graph displays luciferase activation levels relative to transfection with a control vector (pCMV-GFP). Bars represent the average (mean±st.dev.) obtained from three independent transfection experiments.

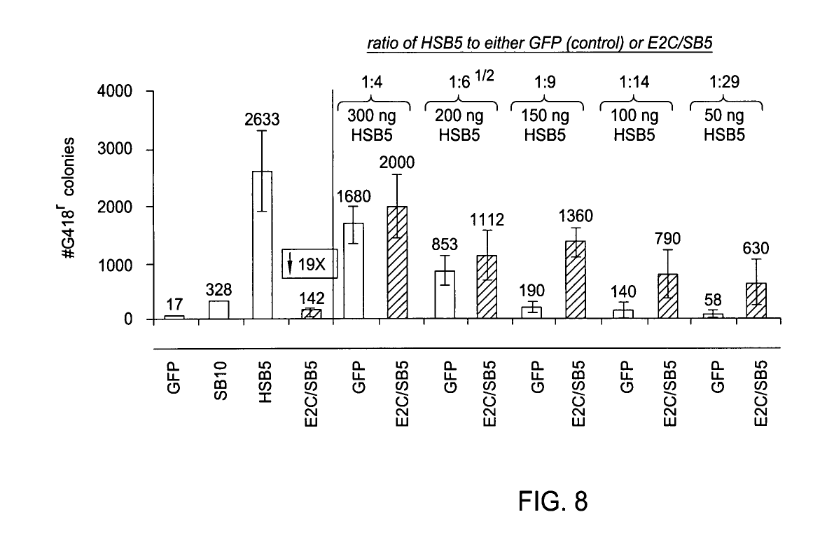

The activity of the E2C/SB-5 fusion protein in a mixture comprising HSB5 transposase was compared to the individual activities of the wild-type SB transposase, the HSB5 transposase, and the E2C/SB-5 fusion protein. HeLa cells were transfected with 1.5 μg of a neomycin-marked transposon (pT/nori) together with a total of 1.5 μg of a helper plasmid, with the helper plasmid being either one plasmid encoding GFP, SB10 transposase, HSB5 transposase, or E2C/SB-5 fusion protein (lanes 1-4 of FIG. 8), or a mixture of two plasmids, with the first plasmid encoding either GFP or E2C/SB-5 fusion protein, and a limiting amount of a second plasmid encoding HSB5 transposase (lanes 5-14). Cells were selected for resistance to the antibiotic G418 for two weeks, at which time individual G418-resistant (G418^{R}) colonies were fixed, stained, and counted. Lanes 13 and 14 show that the expression of 29-fold more E2C/SB-5 fusion protein relative to HSB5 transposase (i.e., 50 ng of HSB5 transposase plasmid and 1.45 μg of E2C/SB-5 fusion protein plasmid) resulted in approximately a 10-fold higher integration frequency, as estimated by the number of G418^{R} colonies (630) compared to cells expressing the same limiting amount of HSB5 transposase alone (58 colonies). Lanes 14 and 4 show that the integration frequency in cells containing both the E2C/SB-5 fusion protein and a limiting amount of HSB5 transposase was about 4-fold (630 vs. 142) higher than in cells containing the E2C/SB-5 fusion protein without the limiting amount of the HSB5 transposase. Thus, co-expressing limiting amounts of the HSB5 transposase with the E2C/SB-5 fusion protein caused a synergistic increase in integration frequencies relative to cells expressing either protein alone. Furthermore, it appears that the transposase fusion proteins described according to embodiments of the invention are capable of functioning within the complex milieu of mammalian cells either alone or as mixed multimers with other transposases, in spite of the tight constraints of the synaptic DNA-protein complex.

Site-Specific DNA Integration Using Transposase Fusion Proteins

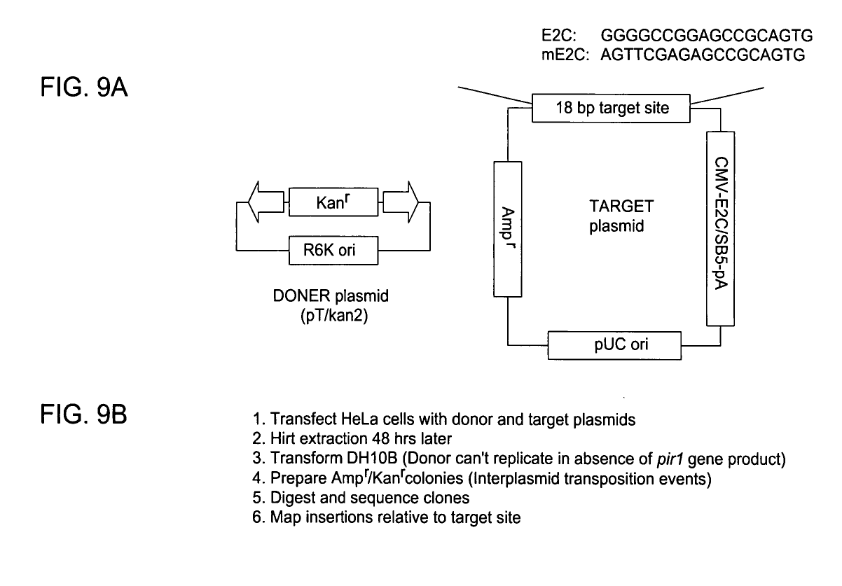

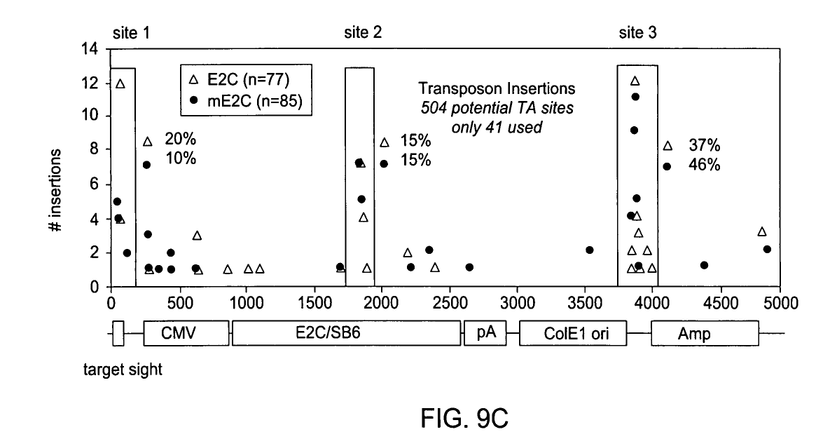

The DNA integration site-specificity of the SB transposase fusion proteins of embodiments of the invention was analyzed by an inter-plasmid transposition assay. A schematic of a donor plasmid and a target plasmid of the assay are shown in FIG. 9A. The donor plasmid (pT/kan2) comprises a kanamycin transposon and an R6K origin of replication that functions only in the presence of the lambda phage pir1 gene product. The target plasmid comprises the Amp^{r} gene, the universal pUC19 origin of replication, and encodes the E2C/SB-5 fusion protein under the control of the CMV promoter. The target plasmid also comprises a single 18 base pair recognition sequence (SEQ ID NO:1) for site-specific binding by either the E2C/SB-5 fusion protein or the unfused E2C protein. Another version of the target plasmid is identical, except that it contains a mutant 18 base pair recognition sequence (SEQ ID NO:2) that is not bound by either the E2C/SB-5 fusion protein or the unfused E2C protein.

FIG. 9B is a list generally summarizing the steps of a strategy to isolate and characterize individual transposition events. In step 1, HeLa cells are transfected with the donor and either of the target plasmids described above with respect to FIG. 9A. In step 2, a Hirt extraction is performed on the cells 48 hours post-transfection to isolate the DNA from the cells. In step 3, the isolated DNA is used to transform the E. coli DH10B. It is noted that the donor plasmid cannot replicate in the absence of the pir1 gene product, and thus, transforming DH10B with the donor plasmid alone should not be sufficient to provide viable transformants. In step 4, the transformants are screened for colonies that are both Amp^{r} and Kan^{r}, i.e., colonies having inter-plasmid transposition events. In step 5, the transformant DNA is digested and sequenced. In step 6, the insertion sites of the donor plasmid in the target plasmid are mapped relative to the target site in the target plasmid.

FIG. 9C shows the integration site distribution of donor plasmids into target plasmids in a transposition assay performed according to the steps described above with respect to FIGS. 9A and 9B. The integration sites in a target plasmid comprising the 18 base pair E2C binding site are shown as diamonds, and the integrations sites in a target plasmid comprising the 18 base pair mutant E2C binding site (mE2C) are shown as circles. Sequence analysis of about 70 transposon insertions for each of the two types of target plasmids indicated that the E2C/SB-5 fusion protein is capable of mediating an entire cycle of DNA transposition, as evidenced by the presence of characteristic target site duplications. Both target plasmids had three regions of frequent transposon insertion and are labeled as sites 1, 2, and 3. Significantly, site 1 is 28 base pairs downstream of the 18 base pair E2C target site, and there were twice as many insertions at site 1 in the target plasmid comprising the unmutated 18 base pair E2C target site compared to the insertions at site 1 in the target plasmid comprising the 18 base pair mutant E2C site (mE2C). This suggests that the E2C/SB-5 fusion protein can bias integration near the E2C binding site via sequence-specific DNA recognition and binding.

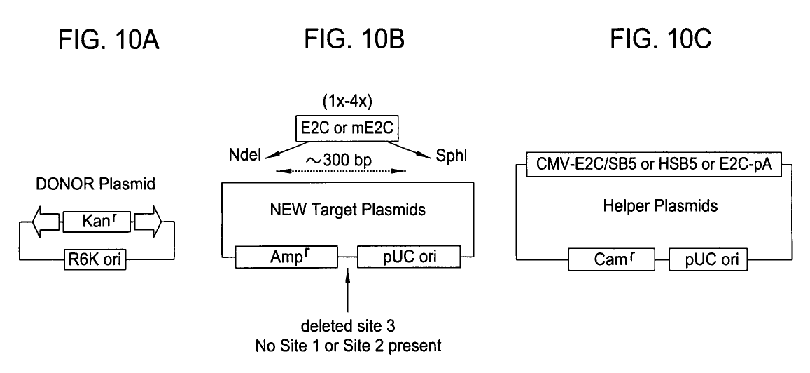

Another embodiment of a transposition assay is provided herein. FIG. 10A is a schematic drawing of a donor plasmid (pT/kan2) comprising a kanamycin transposon and an R6K origin of replication. The donor plasmid may be the same donor plasmid used in the transposition assay described above with respect to FIGS. 9A-9C. FIG. 10B is a schematic drawing of variations of a target plasmid that may be used. The target plasmid comprises the Amp^{r} gene, the pUC19 origin of replication, and from 1 to 4 copies of an unmutated or a mutant E2C binding site in one or both orientations. The 1 to 4 copies of an unmutated or a mutant E2C binding site may be located at either of two different sites in the target plasmid, such as an NdeI site and a SphI site, or at both sites simultaneously. Unlike the target plasmid of FIG. 9C, the target plasmid of FIG. 10B does not include the sequences corresponding to the frequent insertion sites, sites 1-3. Also, the E2C/SB-5 fusion protein is not encoded by the target plasmid as it is in the target plasmid of FIG. 9B. FIG. 10C is a schematic drawing of a helper plasmid that may be used. The helper plasmid encodes the E2C/SB-5 fusion protein under the control of the CMV promoter. Alternatively, the helper plasmid may encode each component of the fusion protein individually (i.e, the HSB5 transposase alone or the E2C protein alone) to control for cis-dependent targeting of integration events by the fusion protein as compared to trans-acting effects, such as bending of the target DNA upon E2C binding. The helper plasmid further comprises the chloramphenicol resistance gene (Cam^{r}) and the pUC19 origin of replication.

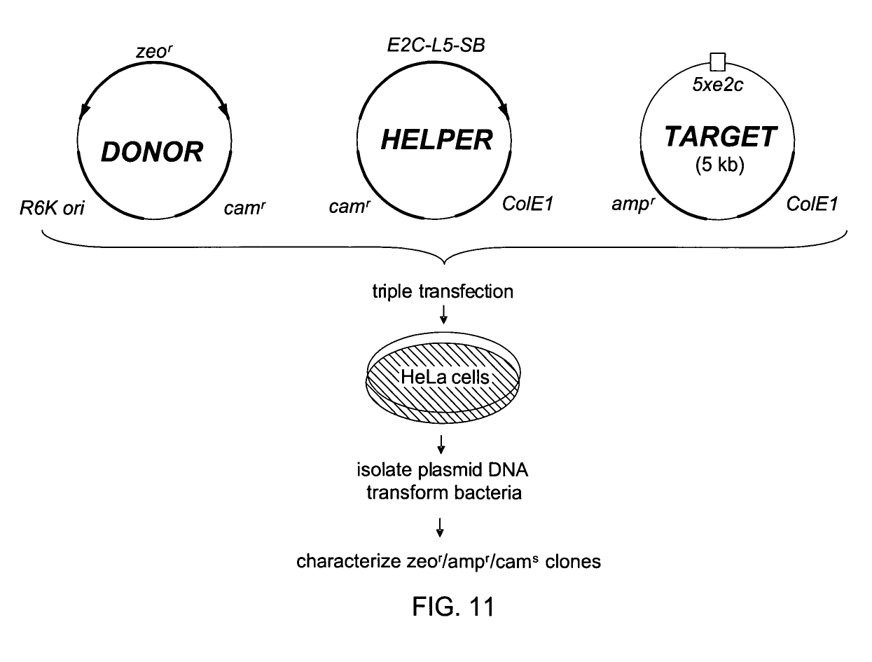

Another embodiment of a plasmid-based transposition assay will be described with respect to FIG. 11. FIG. 11 shows a helper plasmid encoding E2C-L5-SB, a donor plasmid encoding a zeomycin-marked (zeo^{r}) transposon and a counter-selectable chloramphenicol-resistance (cam^{r}) gene, and an ampicillin-resistant (amp^{r}) target plasmid comprising five tandem copies of the e2C recognition sequence. A control helper plasmid encoding an unfused HSB5 as a control is not shown. Replication of the R6K origin-containing donor plasmid is strictly dependent on expression of the pir1 gene product, which is absent from many commonly used bacterial strains, including DH10B. HeLA cells are transfected with the three plasmids. Low-molecular weight plasmid DNA fractions are isolated 2 to 3 days later and used to transform DH10B E. coli. Amp^{r}/zeo^{r} bacteria are first selected, and then patched onto LB-cam^{r} plates to screen for inter-transposition events specific for the target plasmid, i.e., cam^{s}. Pooled and clonal amp^{r}/zeo^{r}/cam^{s} populations of bacteria are then amplified, plasmid DNA is isolated, and the locations of transposon insertions relative to the target sites is determined by restriction site analyses and DNA sequence analyses, respectively.

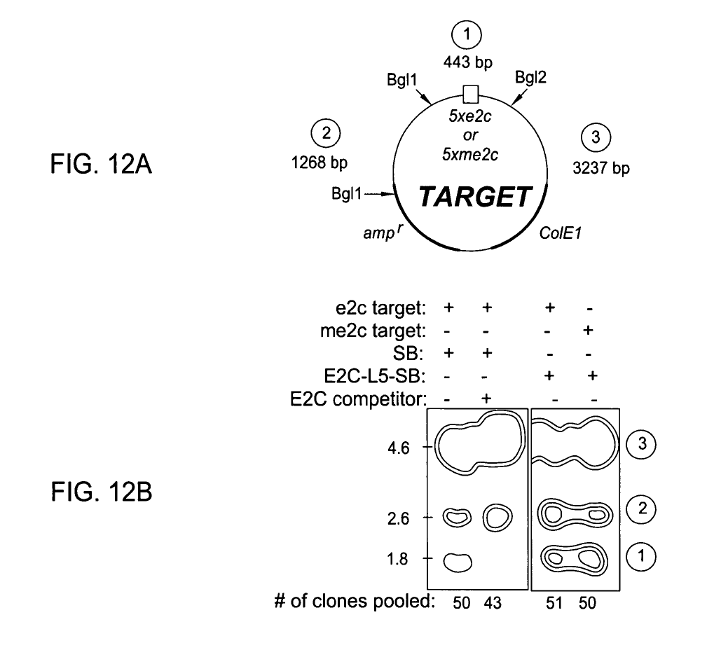

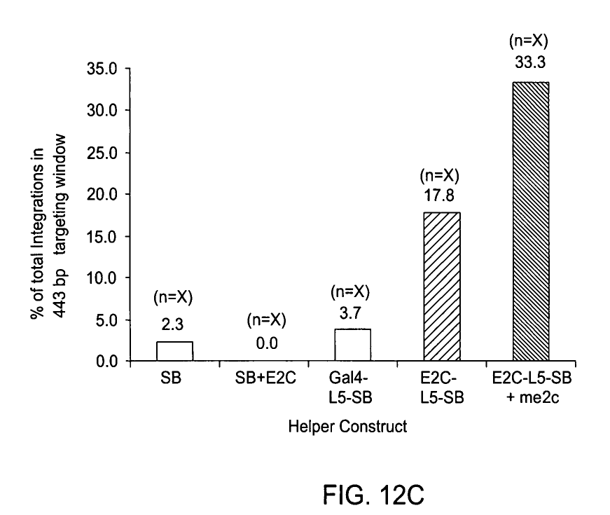

FIG. 12A is an example of a target plasmid that was used in a plasmid-based transposition assay according to FIG. 11. Positions of BglI and BglII restriction endonuclease recognition sites relative to various plasmid features are shown, as are the sizes (in base pairs) for each of the three resulting DNA restriction fragments. FIG. 12B shows the results of DNA blot analysis of targeted integration achieved in an assay using the plasmids of FIGS. 11 and 12A. The assay experimental conditions including using either a target plasmid with the e2C site or the me2C site and a helper plasmid encoding either the HSB5 transposase or the E2C-L5-SB transposase. The effect of a competitor was analyzed by including an E2C competitor, i.e., excess E2C DNA-binding domain was co-expressed with the transposase protein to determine whether associated proteins could inhibit SB target site DNA binding. For each experimental condition, DNA from pooled amp^{r}/zeo^{r}/cam^{s} bacterial colonies (n=43-51) was prepared and treated (500 ng) with BglI/BglII restriction enzymes. Samples were resolved on an agarose gel, transferred to a nitrocellulose membrane, hybridized to a ³²P-radiolabelled probe corresponding to the left SB transposon inverted repeat, and resulting bands visualized upon autoradiography. The lower band intensity, i.e., band 1 in FIG. 12B, under each experimental condition represents a qualitative assessment of the relative frequency of transposition of the 1.35 kb zeo^{r}-marked element into the 443 bp targeting window. FIG. 12C shows the targeted transposition frequencies provided by the different helper plasmids. Recombinant target plasmid DNA was isolated from individual amp^{r}/zeo^{r}/cam^{s} colonies and sequenced using an internal transposon-specific primer. Numbers in parentheses denote the number of integrations analyzed in each group. Bars denote the percentage of total integrations occurring within the 443 bp targeting window.

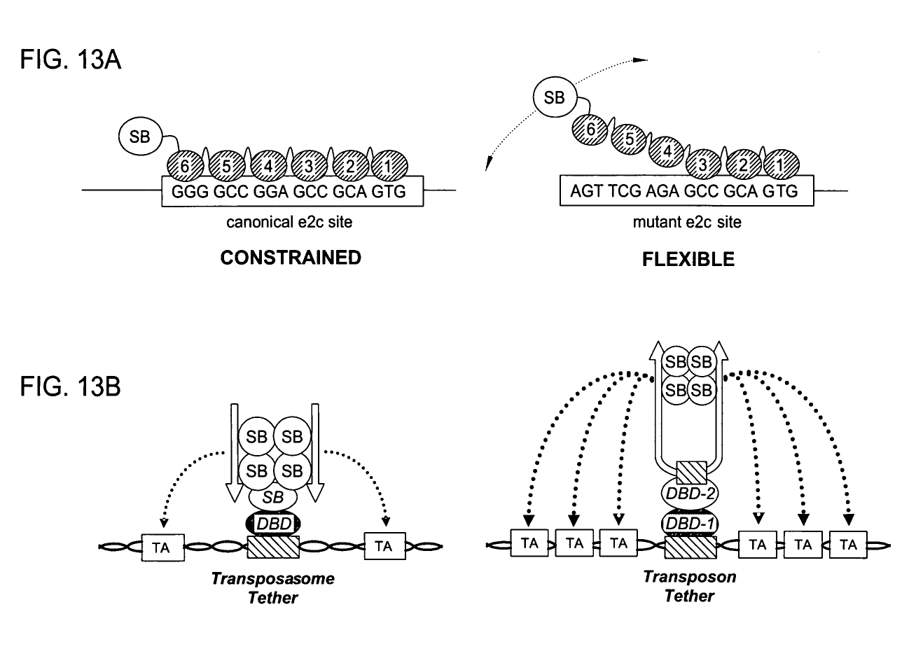

One possible explanation for the higher % of targeted integration of E2C-L5-SB at an me2C site relative to the e2C site as shown in FIG. 12C is that a protein that remains too tightly bound to DNA, such as E2C-L5-SB to the canonical e2C site, cannot efficiently catalyze the multiple changes in both DNA and protein conformation that are necessary to complete a full cycle of transposition. In the case of the mutant e2C site, however, only fingers 1-3 of E2C-L5-SB retain the capacity for DNA-binding (as shown in FIG. 13A), which improves the flexing of the transposase domain, which may enhance the acquisition of and/or manipulation of neighboring target sites.

A transposasome tether approach is shown in FIG. 13B. As shown in FIG. 13B, SB transposase/transposon complexes are tethered to defined target sites via protein-protein interactions. The tether comprises a site-selective DNA-binding domain, such as E2C, fused to SB-G59A-N123, which is unable to bind transposon DNA but still retains the capacity for nuclear retention and subunit multimerization.

A transposon tether approach is also shown in FIG. 13B. Target sites are included within the transposon such that expression of a heterodimeric DNA-binding domain protein (DBD1:DBD2) facilitates tethered transposon flexing and thus regional integration. As shown in the schematic of the transposon tether approach in FIG. 13B, a DNA-binding domain, DBD-1, of the heterodimeric DNA-binding domain protein recognizes the target site in the host genome while the other DNA-binding domain, DBD-2, recognizes the target site in the transposon.

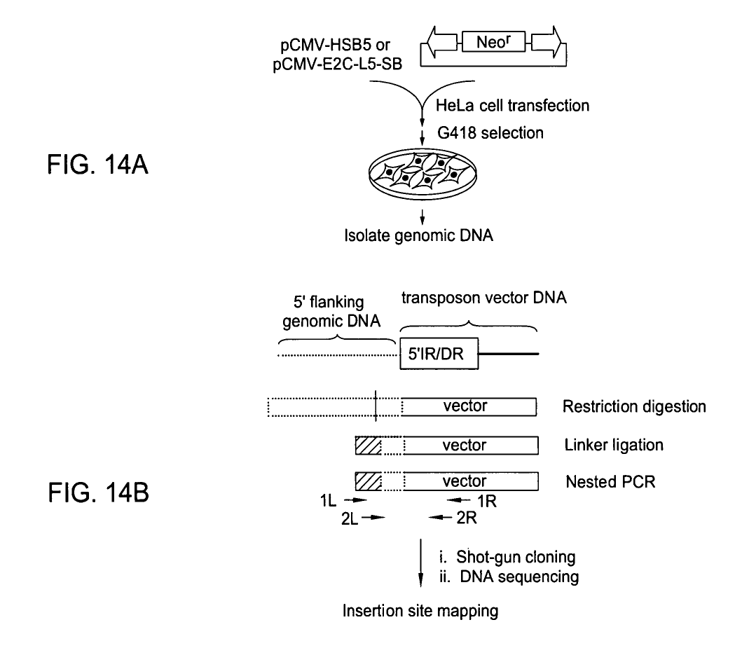

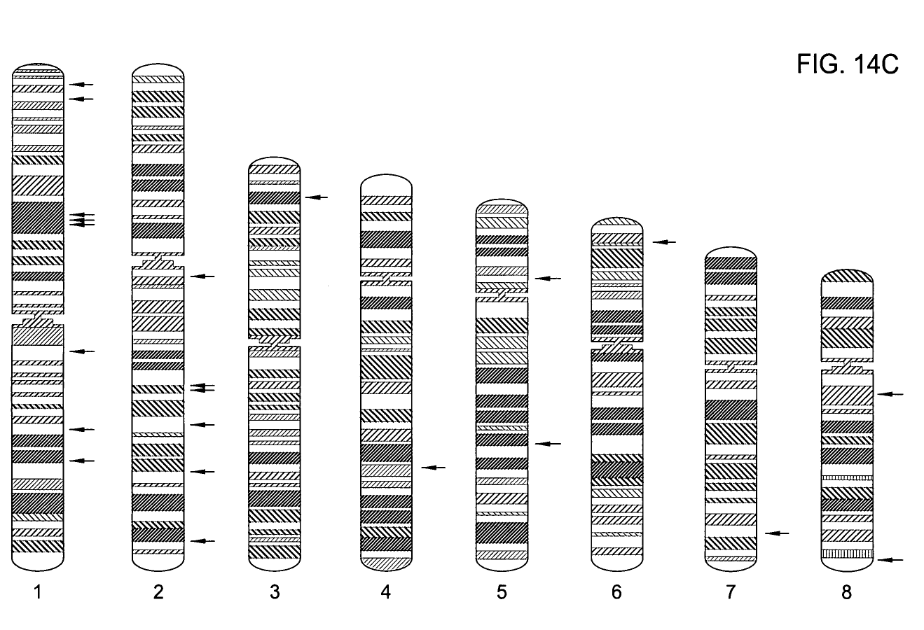

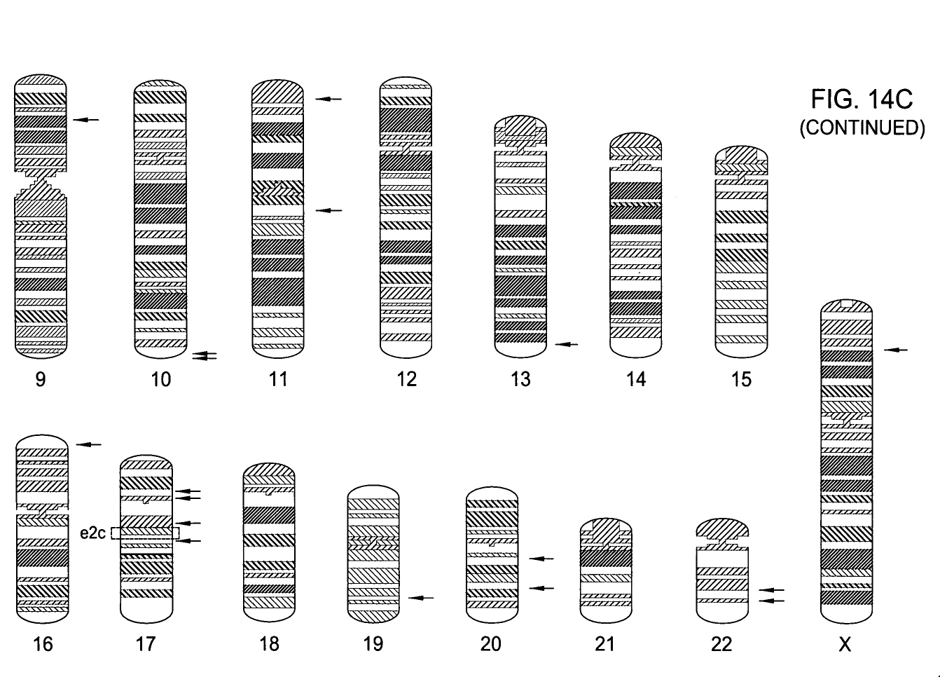

While FIGS. 9A-12C describe inter-plasmid transposition assays, FIGS. 14A-14C describe techniques for and results from using the transposase fusion proteins provided according to embodiments of the invention to mediate site-specific integration in the human genome. As shown in FIG. 14A, transposition into the human genome was initiated by transfecting HeLa cells with plasmids encoding either E2C-L5-SB or HSB5, together with a donor plasmid containing a neomycin-marked (neo^{r}) transposon. Transfected cells were subsequently growth-selected in the antibiotic G418, surviving G418^{r} cells were pooled, and genomic DNA was prepared. FIG. 14B shows a schematic overview of the ligation-mediated polymerase chain reaction (LM-PCR) strategy used to map transposon integrations in the human genome. Genomic DNA samples from G418^{r} pools of cells were digested with BfaI restriction enzyme to release the transposon left inverted repeat, together with short stretches of flanking cellular DNA, from host cell chromosomes. Restricted DNA fragments were ligated to a compatible double-stranded DNA linker and then amplified using two rounds of nested PCR. Amplified fragments were cloned, sequenced using an internal transposon-specific primer, and mapped to the human genome using Ensembl- and BLAST-based homology searches. FIG. 14C shows the distribution of E2C-L5-SB insertion sites in the human genome. The chromosomal positions of X independent insertion events for E2C-L5-SB (arrows) are shown relative to the endogenous e2C site on human chromosome 17 (rectangle). Table 1-summarizes the chromosomal targeting frequency results.

TABLE 1
Chromosomal targeting frequencies in HeLa cells.

| | E2C-L5-SB | SB | |
|---|---|---|---|
| Chrm | n = 67 | n = 55 | EXP |
| 1 | 17.9 | 18.2 | 8.1 |
| 2 | 10.4 | 7.3 | 8.0 |
| 3 | 4.5 | 1.8 | 6.6 |
| 4 | 1.5 | 3.6 | 6.3 |
| 5 | 13.4 | 9.1 | 6.0 |
| 6 | 3.0 | 7.3 | 5.7 |
| 7 | 6.0 | 7.3 | 5.3 |
| 8 | 4.5 | 5.5 | 4.8 |
| 9 | 3.0 | 1.8 | 4.6 |
| 10 | 3.0 | 3.6 | 4.5 |
| 11 | 4.5 | 7.3 | 4.5 |
| 12 | 1.5 | 5.5 | 4.4 |
| 13 | 3.0 | 0.0 | 3.8 |
| 14 | 1.5 | 1.8 | 3.5 |
| 15 | 0.0 | 5.5 | 3.3 |
| 16 | 3.0 | 1.8 | 2.9 |
| 17 | 6.0 | 3.6 | 2.6 |
| 18 | 1.5 | 3.6 | 2.5 |
| 19 | 3.0 | 1.8 | 2.1 |
| 20 | 3.0 | 0.0 | 2.1 |
| 21 | 0.0 | 1.8 | 1.6 |
| 22 | 4.5 | 0.0 | 1.6 |
| X | 1.5 | 1.8 | 5.1 |

While the foregoing is directed to embodiments of the present invention, other and further embodiments of the invention may be devised without departing from the basic scope thereof, and the scope thereof is determined by the claims that follow.

What is claimed is:

1. A fusion protein comprising a transposase.

2. The fusion protein of claim 1, wherein the fusion protein further comprises a site-specific DNA binding protein.

3. A source of transposase activity comprising a fusion protein comprising a Sleeping Beauty transposase and a site-specific DNA binding protein.

4. The source of claim 3, wherein the site-specific DNA binding protein is a zinc-finger DNA binding protein.

5. The source of claim 3, wherein the Sleeping Beauty transposase has the sequence of SEQ ID NO: 17 and the site-specific DNA binding protein comprises the polydactyl zinc finger protein E2C.

6. The source of claim 3, wherein the fusion protein further comprises a flexible linker between the Sleeping Beauty transposase and a site-specific DNA binding protein.

7. A method of integrating an exogenous nucleic acid into a targeted region of a nucleic acid of a mammalian cell, comprising:
introducing a transposon comprising the exogenous nucleic acid and a source of transposase activity into the mammalian cell; and
integrating the exogenous nucleic acid into the targeted region of the nucleic of the mammalian cell.

8. The method of claim 7, wherein the transposon is a Sleeping Beauty transposon, and the transposase is a Sleeping Beauty transposase.

9. The method of claim 8, wherein the source of Sleeping Beauty transposase activity is adapted to recognize the targeted region and integrate the exogenous nucleic acid into the targeted region.

10. The method of claim 9, wherein the source of Sleeping Beauty transposase activity comprises a Sleeping Beauty transposase fused to the polydactyl zinc finger protein E2C.

11. The method of claim 8, wherein the Sleeping Beauty transposon and the source of Sleeping Beauty transposase activity are introduced into the mammalian cell in vitro.

12. The method of claim 8, wherein the Sleeping Beauty transposon and the source of Sleeping Beauty transposase activity are introduced into the mammalian cell in vivo.

13. The method of claim 8, wherein the targeted region is in the genome of the mammalian cell.

14. The method of claim 8, wherein the source of the Sleeping Beauty transposase activity comprises the sequence of SEQ ID NO: 17.

15. The method of claim 8, wherein the source of the Sleeping Beauty transposase activity comprises a fusion protein comprising a Sleeping Beauty transposase and a site-specific DNA binding protein.

16. The method of claim 15, wherein the source of the Sleeping Beauty transposase activity further comprises a hyperactive Sleeping Beauty transposase.


SEQUENCE LISTING (20 sequences)

SEQ ID NO: 1 - DNA, 18 bp, Homo sapiens
ggggccggagccgcagtg

SEQ ID NO: 2 - DNA, 18 bp, Artificial sequence
  Synthetic oligonucleotide containing mutant e2c binding site
agttcgagagccgcagtg

SEQ ID NO: 3 - DNA, 17 bp, Artificial sequence
  Synthetic oligonucleotide containing Gal4 upstream activating sequence
cggagtactgtcctccg

SEQ ID NO: 4 - DNA, 32 bp, Artificial sequence
  Synthetic oligonucleotide containing IDR site
tccagtgggtcagaagtttacatacactaagt

SEQ ID NO: 5 - DNA, 600 bp, Artificial sequence
  Synthetic oligonucleotide representing humanized E2C nucleotide sequence
atggcacaggcagctctggaacccggagagaaaccttatgcctgtcccgaatgtggtaagtccttttctcgaaaagatagccttgtgagacaccagagaacccataccggtgaaaagccttacaagtgcccagagtgcggcaagtctttctcccagtccggggatcttagacggcaccaacgcacccacactggggagaagccatacaaatgtccagagtgtggtaaatccttcagcgactgccgcgacctggcaaggcatcaacgcacacatacaggagaaaagccctacgcttgtcccgaatgcggtaaatctttctctcagtcttcacatcttgtgaggcaccagcgcacacacaccggggagaaaccatataaatgtcctgaatgcggaaagtcttttagcgattgcagggatctcgctagacatcagcgcacccacacaggcgaaaagccttataagtgtccagagtgcggtaaatcctttagcagatccgacaaacttgtacgacaccaaaggacccatactggtaagaaaacaagcggtcaggcaggaggaggttctggcggctccggagggagcggagggtctggagggagc

SEQ ID NO: 6 - protein, 340 aa, Artificial sequence
  Synthetic amino acid representing SB10 transposase protein
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWCGFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 7 - DNA, 1023 bp, Artificial sequence
  Synthetic nucleic acid containing SB10 transposase
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatgagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 8 - DNA, 29 bp, Artificial sequence
  Synthetic nucleic acid containing 5′ outer repeat
gttcaagtcggaagtttacatacacttag

SEQ ID NO: 9 - DNA, 30 bp, Artificial sequence
  Synthetic nucleic acid containing 5′ inner repeat
cagtgggtcagaagtttacatacactaagg

SEQ ID NO: 10 - DNA, 31 bp, Artificial sequence
  Synthetic nucleic acid containing 3′ inner repeat
cagtgggtcagaagttaacatacactcaatt

SEQ ID NO: 11 - DNA, 30 bp, Artificial sequence
  Synthetic nucleic acid containing 3′ outer repeat
agttgaatcggaagtttacatacaccttag

SEQ ID NO: 12 - DNA, 30 bp, Artificial sequence
  Synthetic nucleic acid containing consensus repeat
caktgrgtcrgaagtttacatacacttaag

SEQ ID NO: 13 - DNA, 8 bp, Artificial sequence
  Synthetic nucleic acid containing direct repeat
acatacac

SEQ ID NO: 14 - DNA, 226 bp, Artificial sequence
  Synthetic nucleic acid containing inverted repeat
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 15 - DNA, 228 bp, Artificial sequence
  Synthetic nucleic acid containing inverted repeat
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaatgtatttggctaaggtgtatgtaaacttccgacttcaactg

SEQ ID NO: 16 - DNA, 13 bp, Artificial sequence
  Synthetic nucleic acid containing enhancer element
gtttacagacaga

SEQ ID NO: 17 - protein, 340 aa, Artificial sequence
  Synthetic amino acid containing HSB5 hyperactive SB transposase protein
MGKSKEISQDLRAKIVDLHKSGSSLGAISKRLAVPRSSVQTIVRKYKHHGTTQPSYRSGRRRVLSPRDERTLVRKVQINPRTAAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWCGFAAGGTGALHKIDGIMRKENYVDILKQHLKTSVRKLKLGRKWVFQMDNDPKHTSKVVAKWLKDNKVKVLEWPAQSPDLNPIENLWAELKKRVRARRPTNLTQLHQLCQEEWAKIHPTYCGKLVEGYPKRLTQVKQFKGNATKY

SEQ ID NO: 18 - DNA, 1023 bp, Artificial sequence
  Synthetic nucleic acid containing HSB5 hyperactive SB transposase
atgggaaaatcaaaagaaatcagccaagacctcagagcgaaaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacagcggcaaaggaccttgtgaagatgctggaggaaacaggcacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggtcataatgaccatcgttatgtttggaggaagaagggggaggcttgcaagccgaagaacaccatcccaaccgtgaagcacgggggtggcagcatcatgttgtgggggtgctttgccgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccagcgcaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 19 - protein, 185 aa, Artificial sequence
  Synthetic amino acid representing E2C protein
MAQAALEPGEKPYACPECGKSFSRKDSLVRHQRTHTGEKPYKCPECGKSFSQSGDLRRHQRTHTGEKPYKCPECGKSFSDCRDLARHQRTHTGEKPYACPECGKSFSQSSHLVRHQRTHTGEKPYKCPECGKSFSDCRDLARHQRTHTGEKPYKCPECGKSFSRSDKLVRHQRTHTGKKTSGQAG

SEQ ID NO: 20 - DNA, 558 bp, Artificial sequence
  Synthetic nucleic acid containing E2C nucleotide sequence
atggcccaggcggccctcgagcccggggagaagccctatgcttgtccggaatgtggtaagtccttcagtaggaaggattcgcttgtgaggcaccagcgtacccacacgggtgaaaaaccgtataaatgcccagagtgcggcaaatcttttagtcagtcgggggatcttaggcgtcatcaacgcactcatactggcgagaagccatacaaatgtccagaatgtggcaagtctttcagtgattgtcgtgatcttgcgaggcaccaacgtactcacaccggggagaagccctatgcttgtccggaatgtggtaagtccttctctcagagctctcacctggtgcgccaccagcgtacccacacgggtgaaaaaccgtataaatgcccagagtgcggcaaatcttttagtgactgccgcgaccttgctcgccatcaacgcactcatactggcgagaagccatacaaatgtccagaatgtggcaagtctttcagccgctctgacaagctggtgcgtcaccaacgtactcacaccggtaaaaaaactagtggccaggccggctag